SEQUENCE LISTING (29 sequences)

SEQ ID NO: 1 - protein, 597 aa, Artificial Sequence
  Synthetic
MGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 2 - protein, 590 aa, Artificial Sequence
  Synthetic
MGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 3 - protein, 597 aa, Artificial Sequence
  Synthetic
MGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 4 - protein, 590 aa, Artificial Sequence
  Synthetic
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 5 - protein, 12 aa, Artificial Sequence
  Synthetic
MHHHHHHSSGSS

SEQ ID NO: 6 - protein, 608 aa, Artificial Sequence
  Synthetic
MHHHHHHSSGSSGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 7 - protein, 601 aa, Artificial Sequence
  Synthetic
MHHHHHHSSGSSGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 8 - protein, 608 aa, Artificial Sequence
  Synthetic
MHHHHHHSSGSSGPGQQGPYGPGASAAAAAGQNGPGSGQQGPGQSAAAAAGQYGPGQQGPGQQGPGSSAAAAAGPGQYGPGQQGPSASAAAAAGPGSGQQGPGASAAAAAGQYGPGQQGPGQQGPGSSAAAAAGQYGSGPGQQGPYGSAAAAAGPGSGQYGQGPYGPGASAAAAAGPGQYGPGQQGPSASAAAAAGSGQQGPGQYGPYASAAAAAGQYGSGPGQQGPYGPGQSAAAAAGSGQQGPGQQGPYASAAAAAGPGQQGPYGPGSSAAAAAGQYGYGPGQQGPYGPGASAAAAAGQNGPGSGQYGPGQQGPGQSAAAAAGPGQQGPYGPGASAAAAAGQYGPGQQGPGQYGPGSSAAAAAGPGQQGPYGPGSSAAAAAGQYGPGQQGPYGPGQSAAAAAGQYQQGPGQQGPYGPGASAAAAAGPGQQGPYGPGASAAAAAGPGQYGPGQQGPSASAAAAAGQYGSGPGQYGPYGPGQSAAAAAGPGSGQQGQGPYGPGASAAAAAGQYGPGQQGPYGPGQSAAAAAGPGSGQYGPGASAAAAAGQNGPGSGQYGPGQQGPGQSAAAAAGQYQQGPGQQGPYGPGASAAAAAGPGSGQQGPGAS

SEQ ID NO: 9 - protein, 601 aa, Artificial Sequence
  Synthetic
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGAS

SEQ ID NO: 10 - protein, 565 aa, Artificial Sequence
  Synthetic
MGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 11 - protein, 576 aa, Artificial Sequence
  Synthetic
MHHHHHHSSGSSGPGQQGPYGPGASAAAAAAAGSNGPGSGQQGPGQSGQYGPGQQGPGQQGPGSSAAAAAAAGPGQYGPGQQGPSASAAAAAAAGPGSGQQGPGASGQYGPGQQGPGQQGPGSSAAAAAAAGSYGSGPGQQGPYGSAAAAAAAGPGSGQYGQGPYGPGASGPGQYGPGQQGPSASAAAAAAAGSGQQGPGQYGPYASAAAAAAAGSYGSGPGQQGPYGPGQSGSGQQGPGQQGPYASAAAAAAAGPGQQGPYGPGSSAAAAAAAGSYGYGPGQQGPYGPGASGQNGPGSGQYGPGQQGPGPSAAAAAAAGPGQQGPYGPGASAAAAAAAGSYGPGQQGPGQYGPGSSGPGQQGPYGPGSSAAAAAAAGSYGPGQQGPYGPGPSAAAAAAAGSYQQGPGQQGPYGPGASGPGQQGPYGPGASAAAAAAAGPGQYGPGQQGPSASAAAAAAAGSYGSGPGQYGPYGPGQSGPGSGQQGQGPYGPGASAAAAAAAGSYGPGQQGPYGPGPSAAAAAAAGPGSGQYGPGASGQNGPGSGQYGPGQQGPGPSAAAAAAAGPGSGQQGPGAS

SEQ ID NO: 12 - protein, 2364 aa, Artificial Sequence
  Synthetic
MGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 13 - protein, 2375 aa, Artificial Sequence
  Synthetic
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 14 - protein, 50 aa, Araneus diadematus
SGCDVLVQALLEVVSALVSILGSSSIGQINYGASAQYTQMVGQSVAQALA

SEQ ID NO: 15 - protein, 30 aa, Araneus diadematus
SGCDVLVQALLEVVSALVSILGSSSIGQIN

SEQ ID NO: 16 - protein, 21 aa, Araneus diadematus
SGCDVLVQALLEVVSALVSIL

SEQ ID NO: 17 - protein, 1154 aa, Artificial Sequence
  Synthetic
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVSRARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSIL

SEQ ID NO: 18 - protein, 24 aa, Artificial Sequence
  Synthetic
MHHHHHHHHHHSSGSSLEVLFQGP

SEQ ID NO: 19 - protein, 590 aa, Artificial Sequence; identical to SEQ ID NO: 4
  Synthetic

SEQ ID NO: 20 - protein, 612 aa, Artificial Sequence
  Synthetic
MGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 21 - protein, 565 aa, Artificial Sequence; identical to SEQ ID NO: 10
  Synthetic

SEQ ID NO: 22 - protein, 592 aa, Artificial Sequence
  Synthetic
MGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 23 - protein, 619 aa, Artificial Sequence
  Synthetic
MGPGQQGPYGPGASAAAAAAAGSNGPGSGQQGPGQSGQYGPGQQGPGQQGPGSSAAAAAAAGPGQYVLIGPGQQVLIGPSASAAAAAAAGPGSGQQGPGASGQYGPGQQGPGQQGPGSSAAAAAAAGSYGSVLIGPGQQVLIGPYGSAAAAAAAGPGSGQYGQGPYGPGASGPGQYGPGQQGPSASAAAAAAAGSGQQVLIGPGQYVLIGPYASAAAAAAAGSYGSGPGQQGPYGPGQSGSGQQGPGQQGPYASAAAAAAAGPGQQVLIGPYVLIGPGSSAAAAAAAGSYGYGPGQQGPYGPGASGQNGPGSGQYGPGQQGPGPSAAAAAAAGPGQQVLIGPYVLIGPGASAAAAAAAGSYGPGQQGPGQYGPGSSGPGQQGPYGPGSSAAAAAAAGSYGPGQQVLIGPYVLIGPGPSAAAAAAAGSYQQGPGQQGPYGPGASGPGQQGPYGPGASAAAAAAAGPGQYVLIGPGQQVLIGPSASAAAAAAAGSYGSGPGQYGPYGPGQSGPGSGQQGQGPYGPGASAAAAAAAGSYGPGQQVLIGPYVLIGPGPSAAAAAAAGPGSGQYGPGASGQNGPGSGQYGPGQQGPGPSAAAAAAAGPGSGQQVLIGPGASVLI

SEQ ID NO: 24 - protein, 623 aa, Artificial Sequence
  Synthetic
MHHHHHHSSGSSGPGQQGPYGPGASAAAAAGQNGPGSGQQGPGQSGQYGPGQQGPGQQGPGSSAAAAAGPGQYVLIGPGQQVLIGPSASAAAAAGPGSGQQGPGASGQYGPGQQGPGQQGPGSSAAAAAGSYGSVLIGPGQQVLIGPYGSAAAAAGPGSGQYGQGPYGPGASGPGQYGPGQQGPSASAAAAAGSGQQVLIGPGQYVLIGPYASAAAAAGQYGSGPGQQGPYGPGQSGSGQQGPGQQGPYASAAAAAGPGQQVLIGPYVLIGPGSSAAAAAGQYGYGPGQQGPYGPGASGQNGPGSGQYGPGQQGPGQSAAAAAGPGQQVLIGPYVLIGPGASAAAAAGQYGPGQQGPGQYGPGSSGPGQQGPYGPGSSAAAAAGSYGPGQQVLIGPYVLIGPGPSAAAAAGQYQQGPGQQGPYGPGASGPGQQGPYGPGASAAAAAGPGQYVLIGPGQQVLIGPSASAAAAAGQYGSGPGQYGPYGPGQSGPGSGQQGQGPYGPGASAAAAAGSYGPGQQVLIGPYVLIGPGPSAAAAAGPGSGQYGPGASGQNGPGSGQYGPGQQGPGQSAAAAAGQYQQVLIGPGQQGPYVLIGPGASAAAAAGPGSGQQVLIGPGASVLI

SEQ ID NO: 25 - protein, 603 aa, Artificial Sequence
  Synthetic
MHHHHHHSSGSSGPGQQGPYGPGASAAAAAAAGSNGPGSGQQGPGQSGQYGPGQQGPGQQGPGSSAAAAAAAGPGQYVLIGPGQQGPSASAAAAAAAGPGSGQQGPGASGQYGPGQQGPGQQGPGSSAAAAAAAGSYGSVLIGPGQQGPYGSAAAAAAAGPGSGQYGQGPYGPGASGPGQYGPGQQGPSASAAAAAAAGSGQQVLIGPGQYGPYASAAAAAAAGSYGSGPGQQGPYGPGQSGSGQQGPGQQGPYASAAAAAAAGPGQQVLIGPYGPGSSAAAAAAAGSYGYGPGQQGPYGPGASGQNGPGSGQYGPGQQGPGPSAAAAAAAGPGQQVLIGPYGPGASAAAAAAAGSYGPGQQGPGQYGPGSSGPGQQGPYGPGSSAAAAAAAGSYGPGQQVLIGPYGPGPSAAAAAAAGSYQQGPGQQGPYGPGASGPGQQGPYGPGASAAAAAAAGPGQYVLIGPGQQGPSASAAAAAAAGSYGSGPGQYGPYGPGQSGPGSGQQGQGPYGPGASAAAAAAAGSYGPGQQVLIGPYGPGPSAAAAAAAGPGSGQYGPGASGQNGPGSGQYGPGQQGPGPSAAAAAAAGPGSGQQGPGASVLI

SEQ ID NO: 26 - protein, 559 aa, Artificial Sequence
  Synthetic
MHHHHHHHHHHSSGSSLEVLFQGPGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGPGGSYGPGGAGGPYGPGGPYGPGGEGPGGAGGPYGPGGVGPGGGGPGGYGPGGAGPGGYGPGGSGPGGYGPGGSGPGGYGPGGSGPGGYGPGGSGPGGYGPGGSGPGGSGPGGYGPGGSGPGGSGPGGYGPGGSGPGGYGPGGSGPGGSGPGGYGPGGSGPGGSGPGGYGPGGSGPGGFGPGGFGPGGSGPGGYGPGGSGPGGAGPGGVGPGGFGPGGAGPGGAGPGGAGPGGAGPGGAGPGGAGPGGAGPGGAGPGGAGGAGGAGGAGGSGGAGGSGGTTIIEDLDITIDGADGPITISEELTISAYYPSSRVPDMVNGIMSAMQGSGFNYQMFGNMLSQYSSGSGTCNPNNVNVLMDALLAALHCLSNHGSSSFAPSPTPAAMSAYSNSVGRMFAY

SEQ ID NO: 27 - protein, 252 aa, Artificial Sequence
  Synthetic
MHHHHHHSSGSSKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEK

SEQ ID NO: 28 - protein, 310 aa, Artificial Sequence
  Synthetic
MHHHHHHPEPPVNSYLPPSDSYGAPGQSGPGGRPSDSYGAPGGGNGGRPSDSYGAPGQGQGQGQGQGGYAGKPSDSYGAPGGGDGNGGRPSSSYGAPGGGNGGRPSDTYGAPGGGNGGRPSDTYGAPGGGGNGNGGRPSSSYGAPGQGQGNGNGGRPSSSYGAPGGGNGGRPSDTYGAPGGGNGGRPSDTYGAPGGGNNGGRPSSSYGAPGGGNGGRPSDTYGAPGGGNGNGSGGRPSSSYGAPGQGQGGFGGRPSDSYGAPGQNQKPSDSYGAPGSGNGNGGRPSSSYGAPGSGPGGRPSDSYGPPASG

SEQ ID NO: 29 - protein, 282 aa, Artificial Sequence
  Synthetic
MHHHHHHSSGSSLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVG

(19) United States
(12) Patent Application Publication
(10) Pub. No.: US 2021/0388557 A1
(43) Pub. Date: Dec. 16, 2021

(54) Method for Producing Protein Fiber, and Method for Shrinking Protein Fiber

(71) Applicants: SPIBER INC., Tsuruoka-shi, Yamagata (JP); KOJIMA INDUSTRIES CORPORATION, Toyota-shi, Aichi (JP)

(72) Inventors: Masataka KAJI, Kanazawa-shi, Ishikawa (JP); Ryuhei ENDO, Kanazawa-shi, Ishikawa (JP); Satoshi MiYAGUCHI, Kanazawa-shi, Ishikawa (JP); Kohsuke TOGASHI, Nomi-shi, Ishikawa (JP); Daichi YAMAGAMI, Nomi-shi, Ishikawa (JP); Mizuki IGARASHI, Tsuruoka-shi, Yamagata (JP); Takahito NISHIKADO, Tsuruoka-shi, Yamagata (JP)

(73) Assignees: Spiber Inc., Tsuruoka-shi, Yamagata (JP); Kojima Industries Corporation, Toyota-shi, Aichi (JP)

(21) Appl. No.: 16/491,655

(22) PCT Filed: Mar. 8, 2018
(86) PCT No.: PCT/JP2018/009069

§ 371 Date: Sep. 6, 2019

(30) Foreign Application Priority Data
Mar. 10, 2017 (JP) 2017-046527

(51) Int. Cl. D06M 11/05

(52) U.S. Cl. CPC D06M 11/05; D06M 2101/12

(57) ABSTRACT

The present invention relates to a method for producing a protein fiber, including a step of bringing a protein raw fiber containing a protein into contact with water vapor in a storing chamber in which a temperature is adjusted within a range of less than 120° C. to perform heat treatment of the protein raw fiber.

### TECHNICAL FIELD

The present invention relates to a method for producing a protein fiber, and a method for pre-shrinking a protein fiber.

### BACKGROUND ART

Some protein fibers have a property of contracting upon contact with moisture (for example, immersion in water or hot water, exposure to a high-humidity environment, or the like). This property causes various problems in production process and in commercialization of products.

Under such circumstances, for example, Patent Literature 1 discloses a method for pre-shrinking a silk fabric using high twist yarn that has completed scouring, in which the silk fabric is immersed in water, other solvents, or their mixed system in a tension state, and is heat for a predetermined time. Patent Literature 2 discloses a method for processing silk fibers, which imparts washability and antifouling property to the silk fibers that have been woven and formed into a fabric, and in which the silk fibers are subjected to deterioration preventing treatment using a water soluble cyanuric chloride derivative or a water soluble vinyl sulfone derivative as a crosslinking agent; pre-shrinkage treatment using any of a steaming method, a vacuum steaming method, or a sanforizing method; and water repellent finish treatment using a fluorine-based water repellent finish agent. Patent Literature 3 discloses a method for immobilizing a shape of an animal fiber product, in which the animal fiber product in a state of being formed into a required shape is subjected to water vapor treatment to bringing the product into contact with high-pressure saturated water vapor at 120° C. to 200° C., and thereby the shape of the fiber product is fixed at the time of the water vapor treatment.

### CITATION LIST

### Patent Literature

[Patent Literature 1] Japanese Examined Patent Publication No. H2-6869

[Patent Literature 2] Japanese Unexamined Patent Publication No. 2012-246580

[Patent Literature 3] Japanese Unexamined Patent Publication No. H6-294068

### SUMMARY OF INVENTION

### Problems to be Solved by the Invention

However, the methods disclosed in Patent Literature 1 to Patent Literature 3 relate to the technology for pre-shrinking fiber products, and it is difficult to apply them directly to pre-shrinkage of protein fibers that are materials. In particular, the method disclosed in Patent Literature 3 involves a danger in work because the method handles high-temperature water.

An object of the present invention is to provide a method for producing a protein fiber by which protein fibers in which a contraction amount due to contact with moisture is reduced can be obtained.

Another object of the present invention is to provide a method for pre-shrinking a protein fiber by which a contraction amount due to contact with moisture can be reduced.

### Means for Solving the Problems

The present invention relates to, for example, each of the following inventions.

[1]

A method for producing a protein fiber, including a step of bringing a protein raw fiber containing a protein into contact with water vapor in a storing chamber in which a temperature is adjusted within a range of less than 120° C. to perform heat treatment of the protein raw fiber.

[2]

The method for producing a protein fiber according to [1], in which the heat treatment is performed for 1 minute or longer.

[3]

The method for producing a protein fiber according to [1] or [2], in which the protein is a structural protein.

[4]

The method for producing a protein fiber according to [3], in which the structural protein is a spider silk fibroin.

[5]

The method for producing a protein fiber according to any one of [1] to [4], in which a plurality of the protein raw fibers are bundled together and are twisted.

[6]

The method for producing a protein fiber according to any one of [1] to [5], in which the heat treatment is performed in a state in which the protein raw fiber is not loosened.

[7]

The method for producing a protein fiber according to any one of [1] to [6], in which the heat treatment is performed under reduced pressure.

[8]

A method for producing fabric made of protein fibers, including a step of producing fabric using the protein fiber obtained by the method for producing a protein fiber according to any one of [1] to [7].

[9]

A method for pre-shrinking a protein fiber, including a step of bringing a protein raw fiber containing a protein into contact with water vapor in a storing chamber in which a temperature is adjusted within a range of less than 120° C. to heat-treat the protein raw fiber.

### Effects of the Invention

According to the present invention, it is possible to provide a method for producing a protein fiber by which protein fibers in which a contraction amount due to contact with moisture is reduced can be obtained. The production method of the present invention includes steam setting at less than 120° C., and thereby desired protein fibers can be obtained more safely as compared to the method disclosed in Patent Literature 3.

According to the present invention, it is also possible to provide a method for pre-shrinking a protein fiber by which a contraction amount due to contact with moisture can be reduced.

### BRIEF DESCRIPTION OF DRAWINGS

FIG. 1 is a schematic diagram showing an example of a domain sequence of a modified fibroin.

FIG. 2 is a schematic diagram showing the example of the domain sequence of the modified fibroin.

FIG. 3 is an explanatory view schematically showing an example of a spinning apparatus for producing protein raw fibers.

### EMBODIMENTS FOR CARRYING OUT THE INVENTION

Hereinafter, although preferred embodiments of the present invention will be described in detail with reference to the drawings depending on the case, the present invention is not limited to the following embodiments. In the drawings, the same or corresponding portions are denoted by the same reference numerals, and overlapping descriptions will be appropriately omitted.

[Method for producing protein fiber]

A method for producing a protein fiber according to the present embodiment includes a step of bringing a protein raw fiber containing a protein into contact with water vapor in a storing chamber in which a temperature is adjusted within a range of less than 120° C. to heat-treat the protein raw fiber (hereinafter, will be referred to as the “heat treatment step,” “water vapor heat treatment step,” or “steam setting step”). By passing through the water vapor heat treatment step, a contraction amount of protein fibers thus obtained is reduced (an amount of change in length in a fiber direction) in a case where the fibers come in contact with moisture. The water vapor heat treatment step can be carried out by, for example, after storing protein raw fibers containing a protein in a storage chamber (for example, a storage chamber of a steam setting device), adjusting a temperature of the storage chamber to less than 120° C. by introducing water vapor into the storage chamber, and heat-treating the protein raw fibers.

(Protein)

The protein fibers produced according to the production method of the present invention, or protein raw fibers as raw materials, contain a protein that imparts the fibers contracted upon contact with moisture as a main component. The protein is not particularly limited, and may be a protein produced by a microorganism or the like by genetic recombination technology, or may be a protein produced synthetically, or may be a protein obtained by purifying naturally occurring proteins.

The protein may be, for example, a structural protein and an artificial structural protein derived from the structural protein. The structural protein means a protein which forms or retains the structure, form and the like in vivo. Examples of structural proteins include fibroin, keratin, collagen, elastin, resilin, and the like.

The structural protein may be a fibroin. The fibroin may be, for example, one or more kinds selected from the group consisting of a silk fibroin, a spider silk fibroin, and a hornet silk fibroin. In particular, the structural protein may be a silk fibroin, a spider silk fibroin or a combination thereof. In a case where a silk fibroin and a spider silk fibroin are used in combination, a proportion of silk fibroin may be, for example, 40 parts by mass or less, 30 parts by mass or less, or 10 parts by mass or less with respect to 100 parts by mass of a spider silk fibroin.

A silk thread is a fiber (cocoon yarn) obtained from cocoons made by silkworms which are larvae of silkworms (Bombyx mori). In general, one cocoon thread is composed of two silk fibroins and a shell material (sericin) covering them from the outside. The silk fibroin is composed of a large number of fibrils. The silk fibroin is covered with four layers of sericin. In practice, silk filaments obtained by dissolving and removing outer sericin by scouring are used for clothing applications. A general silk thread has a specific gravity of 1.33, a fineness of 3.3 decitex on average, and a fiber length of about 1300 to 1500 m. The Silk fibroin can be obtained from natural or domestic silkworm cocoons, or used or discarded silk fabrics as a raw material.

As the silk fibroin, a sericin-removed silk fibroin, a sericin-unremoved silk fibroin, or a combination thereof may be used. The sericin-removed silk fibroin is obtained by removing and purifying sericin covering silk fibroin, other fats, and the like. The silk fibroin thus purified is preferably used as a freeze-dried powder. The sericin-unremoved silk fibroin is an unpurified silk fibroin from which sericin and the like are not removed.

The spider silk fibroin may contain a spider silk polypeptide selected from the group consisting of natural spider silk proteins and polypeptides derived from natural spider silk proteins (artificial spider silk proteins).

Examples of natural spider silk proteins include large nasogastric silkworm silk proteins, weft silk proteins, and viallet gland proteins. The large nasogastric silkworm silk has high stress and elasticity because it has a repeated region consisting of a crystalline region and a non-crystallin region (also referred to as an amorphous region). The weft of spider silk has a characteristic of having no crystalline region and having a repeated region consisting of a non-crystallin region. The weft is inferior in stress as compared with the large nasogastric silkworm silk, but has high elasticity.

The large nasogastric silkworm silk protein is characterized by having excellent toughness because of being produced in a large spider line. Examples of large nasogastric silkworm silk proteins include major ampullate spidroins MaSp1 and MaSp2 derived from Nephila clavipes, and ADF3 and ADF4 derived from Araneus diadematus. ADF3 is one of the two major dragline proteins of Araneus diadematus. The polypeptides derived from natural spider silk proteins may be polypeptides derived from these dragline proteins. The polypeptides derived from ADF3 are relatively easily synthesized, and have excellent properties in terms of strength-elongation and toughness.

A weft protein is produced in the flagelliform gland of the spider. Examples of weft proteins include a flagelliform silk protein derived from Nephila clavipes.

A polypeptide derived from a natural spider silk protein may be a recombinant spider silk protein. Examples of recombinant spider silk proteins include mutants, analogues, or derivatives of natural spider silk proteins. A preferred example of such a polypeptide is a recombinant spider silk protein (will be referred to as a “polypeptide derived from the large nasogastric dragline protein”) of the large nasogastric silkworm protein.

Examples of fibroin-like proteins derived from the large nasogastric silkworm and proteins derived from Bombyx mori silk include proteins including domain sequences represented by Formula 1: [(A)ₙ motif-REP]ₘ or Formula 2: [(A)ₙ motif-REP]ₘ−(A)ₙ motif. The (A)ₙ motif indicates an amino acid sequence mainly including an alanine residue, and the number of amino acid residues is 2 to 27. The number of amino acid residues of the (A)ₙ motif may be an integer of 2 to 20, 4 to 27, 4 to 20, 8 to 20, 10 to 20, 4 to 16, 8 to 16, or 10 to 16. In addition, it is sufficient as long as a ratio of the number of alanine residues with respect to the total number of amino acid residues in the (A)ₙ motif is 40% or more, and it may be 60% or more, 70% or more, 80% or more, 83% or more, 85% or more, 86% or more, 90% or more, 95% or more, or 100% (meaning it consists only of alanine residues). At least seven of a plurality of (A)ₙ motifs present in the domain sequence consist of only alanine residues. REP represents an amino acid sequence consisting of 2 to 200 amino acid residues. REP may be an amino acid sequence consisting of 10 to 200 amino acid residues. m indicates an integer of 2 to 300, and may be an integer of 10 to 300. A plurality of (A)ₙ motifs may be the same amino acid sequence or different amino acid sequences. A plurality of REPs may be the same amino acid sequence or different amino acid sequences.

A modified fibroin derived from the large nasogastric silkworm silk protein produced in the large ampullate gland of the spider includes a unit of the amino acid sequence represented by Formula 1: [(A)ₙ motif-REP]ₘ, and may be a polypeptide which is an amino acid sequence having a homology of 90% or more to the amino acid sequence whose C-terminal sequence is shown in any of SEQ ID NOs: 14 to 16, or the amino acid sequence set forth in any of SEQ ID NOs: 14 to 16.

The amino acid sequence set forth in SEQ ID NO: 14 is identical to the amino acid sequence consisting of 50 amino acid residues at the C-terminus of the amino acid sequence of ADF3 (GI: 1263287, NCBI); the amino acid sequence set forth in SEQ ID NO: 15 is identical to the amino acid sequence obtained by removing 20 residues from the C-terminus of the amino acid sequence set forth in SEQ ID NO: 14; and the amino acid sequence set forth in SEQ ID NO: 16 is identical to the amino acid sequence obtained by removing 29 residues from the C-terminus of the amino acid sequence set forth in SEQ ID NO: 14.

A more specific example of the modified fibroin derived from the large nasogastric silkworm silk protein produced in the large ampullate gland of the spider may be a modified fibroin including (1-i) an amino acid sequence set forth in SEQ ID NO: 17 or (1-ii) an amino acid sequence having 90% or more sequence identity with the amino acid sequence set forth in SEQ ID NO: 17. It is preferred that the sequence identity is 95% or more.

The amino acid sequence set forth in SEQ ID NO: 17 is an amino acid sequence in which the first to thirteenth repeat regions are increased to approximately double, and which is mutated so that the translation is terminated at the 1154th amino acid residue, in the amino acid sequence of ADF3 in which the amino acid sequence consisting of initiation codon, His10 tag, and HRV3C protease (Human rhinovirus 3C protease) recognition site (SEQ ID NO: 18) are added to the N terminus. The amino acid sequence at the C-terminus of the amino acid sequence set forth in SEQ ID NO: 17 is identical to the amino acid sequence set forth in SEQ ID NO: 16.

The modified fibroin of (1-i) may consist of the amino acid sequence set forth in SEQ ID NO: 17.

The modified fibroin in which a content of glycine residues is reduced has an amino acid sequence whose domain sequence has a reduced content of glycine residues as compared to naturally occurring fibroin. The modified fibroin can be said to have at least an amino acid sequence corresponding to substitution of one or a plurality of glycine residues in REP with another amino acid residue, as compared to naturally occurring fibroin.

The modified fibroin in which a content of glycine residues is reduced may be a modified fibroin in which the domain sequence has, in at least one motif sequence selected from GGX and GPGXX (where G represents a glycine residue, P represents a proline residue, and X represents an amino acid residue other than glycine) in REP, at least an amino acid sequence corresponding to substitution of one glycine residue in one or a plurality of the motif sequences with another amino acid residue, as compared to the naturally occurring fibroin.

The modified fibroin in which a content of glycine residues is reduced may be a modified fibroin in which the ratio of the motif sequence in which the glycine residue is substituted with another amino acid residue is 10% or more with respect to the entire motif sequence.

The modified fibroin in which a content of glycine residues is reduced may be a modified fibroin which includes a domain sequence represented by Formula 1: [(A)ₙ motif-REP]ₘ, and has an amino acid sequence in which z/w is 30% or more, 40% or more, 50% or more, or 50.9% or more, in the case where the total number of amino acid residues in the amino acid sequence consisting of XGX (where G represents a glycine residue, and X represents an amino acid residue other than glycine) contained in all REPs in the sequence excluding the sequence from the (A)ₙ motif located at the most C-terminal side to the C-terminus of the domain sequence from the domain sequence is defined as z, and the total number of amino acid residues in the sequence excluding the sequence from the (A)ₙ motif located at the most C-terminal side to the C-terminus of the domain sequence from the domain sequence is defined as w. It is sufficient as long as the number of alanine residues is 83% or more relative to the total number of amino acid residues in the (A)ₙ motif, but it is preferably 86% or more, more preferably 90% or more, still more preferably 95% or more, and even still more preferably 100% (which means that the (A)ₙ motif consists of only alanine residues).

The modified fibroin in which a content of glycine residues is reduced is preferably a modified fibroin in which the content ratio of the amino acid sequence consisting of XGX is increased by substituting one glycine residue of the GGX motif with another amino acid residue. In the modified fibroin in which a content of glycine residues is reduced, the content ratio of the amino acid sequence consisting of GGX in the domain sequence is preferably 30% or less, more preferably 20% or less, still more preferably 10% or less, even still more preferably 6% or less, still further preferably 4% or less, and particularly preferably 2% or less. The content ratio of the amino acid sequence consisting of GGX in the domain sequence can be calculated by the same method as the calculation method of the content ratio (z/w) of the amino acid sequence consisting of XGX described below.

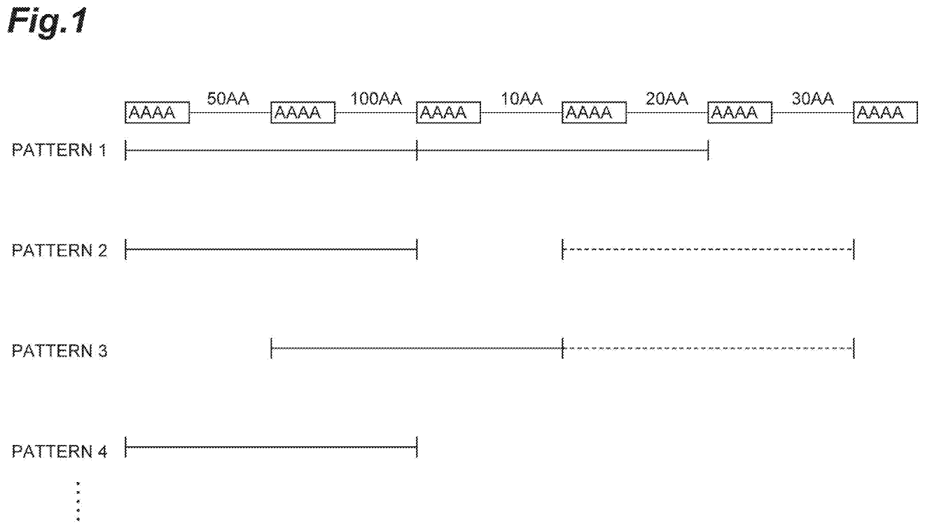

The calculation method of z/w will be described in more detail. First, in a fibroin (a modified fibroin or naturally occurring fibroin) containing a domain sequence represented by Formula 1: [(A)ₙ motif-REP]ₘ from the domain sequence, an amino acid sequence consisting of XGX is extracted from all the REPs contained in the sequence excluding the sequence from the (A)ₙ motif located at the most C-terminal side to the C-terminus of the domain sequence. The total number of amino acid residues constituting XGX is z. For example, in the case where 50 amino acid sequences consisting of XGX are extracted (there is no overlap), z is 50×3=150. Also, for example, in the case where X (central X) contained in two XGXs exists as in the case of the amino acid sequence consisting of XGXGX, it is calculated by subtracting the overlapping portion (in the case of XGXGX, it is 5 amino acid residues). w is the total number of amino acid residues contained in the sequence excluding the sequence from the (A)ₙ motif located at the most C-terminal side to the C-terminus of the domain sequence from the domain sequence. For example, in the case of the domain sequence shown in FIG. 1, w is 4+50+4+100+4+10+4+20+4+30=230 (excluding the (A)ₙ motif located at the most C-terminal side). Next, z/w (%) can be calculated by dividing z by w.

In the modified fibroin in which a content of glycine residues is reduced, z/w is preferably 50.9% or more, more preferably 56.1% or more, still more preferably 58.7% or more, even still more preferably 70% or more, and still further preferably 80% or more. The upper limit of z/w is not particularly limited, but it may be 95% or less, for example.

The modified fibroin in which a content of glycine residues is reduced can be obtained, for example, by substituting and modifying at least a part of a base sequence encoding a glycine residue from the gene sequence of cloned naturally occurring fibroin so as to encode another amino acid residue. At this time, one glycine residue in the GGX motif and GPGXX motif may be selected as the glycine residue to be modified, and substitution may be carried out such that z/w is 50.9% or more. Alternatively, the modified fibroin according to the embodiment can also be obtained, for example, by designing an amino acid sequence satisfying each of the above embodiments from the amino acid sequence of naturally occurring fibroin and chemically synthesizing a nucleic acid encoding the designed amino acid sequence. In any case, in addition to the modification corresponding to substitution of a glycine residue in REP with another amino acid residue from the amino acid sequence of naturally occurring fibroin, modification of the amino acid sequence corresponding to substitution, deletion, insertion and/or addition of one or a plurality of amino acid residues may be carried out.

The above-mentioned another amino acid residue is not particularly limited as long as it is an amino acid residue other than a glycine residue, but it is preferably a hydrophobic amino acid residue such as a valine (V) residue, a leucine (L) residue, an isoleucine (I) residue, a methionine (M) residue, a proline (P) residue, a phenylalanine (F) residue, or a tryptophan (W) residue, or a hydrophilic amino acid residue such as a glutamine (Q) residue, an asparagine (N) residue, a serine (S) residue, a lysine (K) residue, or a glutamic acid (E) residue, among which more preferred is a valine (V) residue, a leucine (L) residue, an isoleucine (I) residue or a glutamine (Q) residue, and still more preferred is a glutamine (Q) residue.

A more specific example of the modified fibroin in which a content of glycine residues is reduced may be a modified fibroin including (2-i) an amino acid sequence set forth in SEQ ID NO: 3, SEQ ID NO: 4, SEQ ID NO: 10, or SEQ ID NO: 12; or (2-ii) an amino acid sequence having 90% or more sequence identity with the amino acid sequence set forth in SEQ ID NO: 3, SEQ ID NO: 4, SEQ ID NO: 10, or SEQ ID NO: 12.

The modified fibroin of (2-i) will be described. The amino acid sequence set forth in SEQ ID NO: 3 is obtained by substituting GQX for all GGX in REP of the amino acid sequence set forth in SEQ ID NO: 1 corresponding to naturally occurring fibroin. The amino acid sequence set forth in SEQ ID NO: 4 is obtained by deleting the (A)ₙ motif every other two positions from the N-terminal side to the C-terminal side from the amino acid sequence set forth in SEQ ID NO: 3 and further inserting one [(A)ₙ motif-REP] before the C-terminal sequence. The amino acid sequence set forth in SEQ ID NO: 10 is obtained by inserting two alanine residues at the C-terminal side of each (A)ₙ motif of the amino acid sequence set forth in SEQ ID NO: 4 and further substituting a part of glutamine (Q) residues with a serine (S) residue to delete a part of amino acids on the N-terminal side so as to be almost the same as the molecular weight of SEQ ID NO: 4. The amino acid sequence set forth in SEQ ID NO: 12 is an amino acid sequence in which a His tag has been added to the C-terminus of a sequence obtained by repeating, 4 times, the region of the 20 domain sequences present in the amino acid sequence set forth in SEQ ID NO: 9 (however, several amino acid residues at the C-terminal side of the region are substituted).

The value of z/w in the amino acid sequence set forth in SEQ ID NO: 1 (corresponding to naturally occurring fibroin) is 46.8%. The values of z/w in the amino acid sequence set forth in SEQ ID NO: 3, the amino acid sequence set forth in SEQ ID NO: 4, the amino acid sequence set forth in SEQ ID NO: 10, and the amino acid sequence set forth in SEQ ID NO: 12 are respectively 58.7%, 70.1%, 66.1%, and 70.0%. In addition, the values of x/y at the Giza ratio (to be described later) 1:1.8 to 1:11.3 of the amino acid sequences set forth in SEQ ID NO: 1, SEQ ID NO: 3, SEQ ID NO: 4, SEQ ID NO: 10, and SEQ ID NO: 12 are respectively 15.0%, 15.0%, 93.4%, 92.7%, and 89.3%.

The modified fibroin of (2-i) may consist of an amino acid sequence set forth in SEQ ID NO: 3, SEQ ID NO: 4, SEQ ID NO: 10, or SEQ ID NO: 12.

The modified fibroin of (2-ii) includes an amino acid sequence having 90% or more sequence identity with the amino acid sequence set forth in SEQ ID NO: 3, SEQ ID NO: 4, SEQ ID NO: 10, or SEQ ID NO: 12. The modified fibroin of (2-ii) is also a protein including a domain sequence represented by Formula 1: [(A)ₙ motif-REP]ₘ. The sequence identity is preferably 95% or more.

It is preferred that the modified fibroin of (2-ii) has 90% or more sequence identity with the amino acid sequence set forth in SEQ ID NO: 3, SEQ ID NO: 4, SEQ ID NO: 10, or SEQ ID NO: 12, and z/w is 50.9% or more in the case where the total number of amino acid residues in the amino acid sequence consisting of XGX (where G represents a glycine residue, and X represents an amino acid residue other than glycine) contained in REP is defined as z, and the total number of amino acid residues of REP in the domain sequence is defined as w.

The above-mentioned modified fibroin may include a tag sequence at either or both of the N-terminus and C-terminus. This makes it possible to isolate, immobilize, detect and visualize the modified fibroin.

The tag sequence may be, for example, an affinity tag utilizing specific affinity (binding property, affinity) with another molecule. As a specific example of the affinity tag, a histidine tag (His tag) can be mentioned. The His tag is a short peptide in which about 4 to 10 histidine residues are arranged and has a property of specifically binding to a metal ion such as nickel, so it can be used for isolation of modified fibroin by chelating metal chromatography. A specific example of the tag sequence may be an amino acid sequence set forth in SEQ ID NO: 5 (amino acid sequence including His tag).

In addition, a tag sequence such as glutathione-S-transferase (GST) that specifically binds to glutathione or a maltose binding protein (MBP) that specifically binds to maltose can also be used.

Further, an “epitope tag” utilizing an antigen-antibody reaction can also be used. By adding a peptide (epitope) showing antigenicity as a tag sequence, an antibody against the epitope can be bound. Examples of the epitope tag include an HA (peptide sequence of hemagglutinin of influenza virus) tag, a myc tag, and a FLAG tag. The modified fibroin can easily be purified with high specificity by utilizing an epitope tag.

It is also possible to use a tag sequence which can be cleaved with a specific protease. By treating a protein adsorbed through the tag sequence with protease, it is also possible to recover the modified fibroin cleaved from the tag sequence.

A more specific example of the modified fibroin including the tag sequence may be a modified fibroin including (2-iii) an amino acid sequence set forth in SEQ ID NO: 8, SEQ ID NO: 9, SEQ ID NO: 11, or SEQ ID NO: 13; or (2-iv) an amino acid sequence having 90% or more sequence identity with the amino acid sequence set forth in SEQ ID NO: 8, SEQ ID NO: 9, SEQ ID NO: 11, or SEQ ID NO: 13.

The amino acid sequences set forth in SEQ ID NO: 6, SEQ ID NO:7, SEQ ID NO: 8, SEQ ID NO: 9, SEQ ID NO: 11, and SEQ ID NO: 13 are amino acid sequences in which an amino acid sequence set forth in SEQ ID NO: 5 (including a His tag sequence and a hinge sequence) is added at the N-terminus of the amino acid sequences set forth in SEQ ID NO: 1, SEQ ID NO: 2, SEQ ID NO: 3, SEQ ID NO: 4, SEQ ID NO: 10, and SEQ ID NO: 12, respectively.

The modified fibroin of (2-iii) may consist of an amino acid sequence set forth in SEQ ID NO: 8, SEQ ID NO: 9, SEQ ID NO: 11, or SEQ ID NO: 13.

The modified fibroin of (2-iv) includes an amino acid sequence having 90% or more sequence identity with the amino acid sequence set forth in SEQ ID NO: 8, SEQ ID NO: 9, SEQ ID NO: 11, or SEQ ID NO: 13. The modified fibroin of (2-iv) is also a protein including a domain sequence represented by Formula 1: [(A)ₙ motif-REP]ₘ. The sequence identity is preferably 95% or more.

It is preferred that the modified fibroin of (2-iv) has 90% or more sequence identity with the amino acid sequence set forth in SEQ ID NO: 8, SEQ ID NO: 9, SEQ ID NO: 11, or SEQ ID NO: 13, and z/w is 50.9% or more in the case where the total number of amino acid residues in the amino acid sequence consisting of XGX (where G represents a glycine residue, and X represents an amino acid residue other than glycine) contained in REP is defined as z, and the total number of amino acid residues of REP in the domain sequence is defined as w.

The above-mentioned modified fibroin may include a secretory signal for releasing the protein produced in the recombinant protein production system to the outside of a host. The sequence of the secretory signal can be appropriately set depending on the type of the host.

The modified fibroin in which a content of (A)ₙ motifs is reduced has an amino acid sequence whose domain sequence has a reduced content of (A)ₙ motifs as compared to naturally occurring fibroin. The domain sequence of the modified fibroin can be said to have at least an amino acid sequence corresponding to deletion of one or a plurality of (A)ₙ motifs, as compared to naturally occurring fibroin.

The modified fibroin in which a content of (A)ₙ motifs is reduced may have an amino acid sequence corresponding to 10 to 40% deletion of the (A)ₙ motif from naturally occurring fibroin.

The modified fibroin in which a content of (A)ₙ motifs is reduced may be a modified fibroin whose domain sequence has at least an amino acid sequence corresponding to deletion of one (A)ₙ motif per one to three (A)ₙ motifs from the N-terminal side to the C-terminal side, as compared to naturally occurring fibroin.

The modified fibroin in which a content of (A)ₙ motifs is reduced may be a modified fibroin whose domain sequence has at least an amino acid sequence corresponding to repetition of two consecutive (A)ₙ motif deletions and one (A)ₙ motif deletion in this order from the N-terminal side to the C-terminal side, as compared to the naturally occurring fibroin.

The modified fibroin in which a content of (A)ₙ motifs is reduced may be a modified fibroin whose domain sequence has at least an amino acid sequence corresponding to deletion of the (A)ₙ motif every other two positions from the N-terminal side to the C-terminal side.

The modified fibroin in which a content of (A)ₙ motifs is reduced may be a modified fibroin which have a domain sequence represented by Formula 1: [(A)ₙ motif-REP]ₘ, and have an amino acid sequence in which x/y is 20% or more, 30% or more, 40% or more, or 50% or more, in the case where the number of amino acid residues in REPs of two adjacent [(A)ₙ motif-REP] units is sequentially compared from the N-terminal side to the C-terminal side, and the number of amino acid residues in REP having a smaller number of amino acid residues is defined as 1, the maximum value of the total value of the number of amino acid residues in the two adjacent [(A)ₙ motif-REP] units where the ratio of the number of amino acid residues in the other REP is 1.8 to 11.3 is defined as x, and the total number of amino acid residues of the domain sequence is y. It is sufficient as long as the number of alanine residues is 83% or more relative to the total number of amino acid residues in the (A)ₙ motif, but it is preferably 86% or more, more preferably 90% or more, still more preferably 95% or more, and even still more preferably 100% (which means that the (A)ₙ motif consists of only alanine residues).

A method of calculating x/y will be described in more detail with reference to FIG. 1. FIG. 1 shows a domain sequence excluding N-terminal sequence and C-terminal sequence from modified fibroin. This domain sequence has a sequence of (A)ₙ motif-first REP (50 amino acid residues)-(A)ₙ motif-second REP (100 amino acid residues)-(A)ₙ motif-third REP (10 amino acid residues)-(A)ₙ motif-fourth REP (20 amino acid residues)-(A)ₙ motif-fifth REP (30 amino acid residues)-(A)ₙ motif from the N-terminal side (left side).

The two adjacent [(A)ₙ motif-REP] units are sequentially selected from the N-terminal side to the C-terminal side so as not to overlap. At this time, an unselected [(A)ₙ motif-REP] unit may exist. FIG. 1 shows a pattern 1 (a comparison between first REP and second REP and a comparison between third REP and fourth REP), a pattern 2 (a comparison between first REP and second REP and a comparison between fourth REP and fifth REP), a pattern 3 (a comparison between second REP and third REP and a comparison between fourth REP and fifth REP), and a pattern 4 (a comparison between first REP and second REP). There are other selection methods besides this.

Next, for each pattern, the number of amino acid residues of each REP in the selected two adjacent [(A)ₙ motif-REP] units is compared. The comparison is carried out by obtaining the ratio of the number of amino acid residues of the other REP in the case where one REP having a smaller number of amino acid residues is 1. For example, in the case of comparing the first REP (50 amino acid residues) and the second REP (100 amino acid residues), the ratio of the number of amino acid residues of the second REP is 100/50=2 in the case where the first REP having a smaller number of amino acid residues is 1. Similarly, in the case of comparing the fourth REP (20 amino acid residues) and the fifth REP (30 amino acid residues), the ratio of the number of amino acid residues of the fifth REP is 30/20=1.5 in the case where the fourth REP having a smaller number of amino acid residues is 1.

In FIG. 1, a set of [(A)ₙ motif-REP] units in which the ratio of the number of amino acid residues of the other REP is 1.8 to 11.3 in the case where one REP having a smaller number of amino acid residues is 1 is indicated by a solid line. Hereinafter, such a ratio is referred to as a Giza ratio. A set of [(A)ₙ motif-REP] units in which the ratio of the number of amino acid residues of the other REP is less than 1.8 or more than 11.3 in the case where one REP having a smaller number of amino acid residues is 1 is indicated by a broken line.

In each pattern, the number of all amino acid residues of two adjacent [(A)ₙ motif-REP] units indicated by solid lines (including not only the number of amino acid residues of REP but also the number of amino acid residues of (A)ₙ motif) is combined. Then, the total values thus combined are compared and the total value of the pattern whose total value is the maximum (the maximum value of the total value) is defined as x. In the example shown in FIG. 1, the total value of the pattern 1 is the maximum.

Next, x/y (%) can be calculated by dividing x by the total amino acid residue number y of the domain sequence.

In the modified fibroin in which a content of (A)ₙ motifs is reduced, x/y is preferably 50% or more, more preferably 60% or more, still more preferably 65% or more, even still more preferably 70% or more, still further preferably 75% or more, and particularly preferably 80% or more. The upper limit of x/y is not particularly limited, and it may be 100% or less, for example. In a case where a Giza ratio is 1:1.9 to 1:11.3, x/y is preferably 89.6% or more; in a case where a Giza ratio is 1:1.8 to 1:3.4, x/y is preferably 77.1% or more; in a case where a Giza ratio is 1:1.9 to 1:8.4, x/y is preferably 75.9% or more; and in a case where a Giza ratio is 1:1.9 to 1:4.1, x/y is preferably 64.2% or more.

In a case where the modified fibroin in which a content of (A)ₙ motifs is reduced is a modified fibroin in which at least seven of (A)n motifs which are present in plural in the domain sequence are composed of only alanine residues, x/y is preferably 46.4% or more, is more preferably 50% or more, is even more preferably 55% or more, is still even more preferably 60% or more, is still even more preferably 70% or more, and is particularly preferable 80% or more. The upper limit of x/y is not particularly limited, and may be 100% or less.

The modified fibroin in which a content of (A)ₙ motifs is reduced can be obtained, for example, from a gene sequence of cloned naturally occurring fibroin, by deleting one or a plurality of the sequences encoding the (A)ₙ motif such that x/y is 64.2% or more. Further, the modified fibroin including a domain sequence with a reduced (A)ₙ motif content can also be obtained, for example, by designing an amino acid sequence corresponding to deletion of one or a plurality of (A)ₙ motifs such that x/y is 64.2% or more, from the amino acid sequence of naturally occurring fibroin, and chemically synthesizing a nucleic acid encoding the designed amino acid sequence. In any case, in addition to the modification corresponding to deletion of (A)ₙ motif from the amino acid sequence of naturally occurring fibroin, modification of the amino acid sequence corresponding to substitution, deletion, insertion and/or addition of one or a plurality of amino acid residues may be carried out.

A more specific example of the modified fibroin in which a content of (A)ₙ motifs is reduced may be a modified fibroin including (3-i) an amino acid sequence set forth in SEQ ID NO: 2, SEQ ID NO: 4, SEQ ID NO: 10, or SEQ ID NO: 12; or (3-ii) an amino acid sequence having 90% or more sequence identity with the amino acid sequence set forth in SEQ ID NO: 2, SEQ ID NO: 4, SEQ ID NO: 10, or SEQ ID NO: 12.

The modified fibroin of (3-i) will be described. The amino acid sequence set forth in SEQ ID NO: 2 is obtained by deleting the (A)ₙ motif every other two positions from the N-terminal side to the C-terminal side from the amino acid sequence set forth in SEQ ID NO: 1 corresponding to naturally occurring fibroin and further inserting one [(A)ₙ motif-REP] before the C-terminal sequence. The amino acid sequence set forth in SEQ ID NO: 4 is obtained by substituting GQX for all GGX in REP of the amino acid sequence set forth in SEQ ID NO: 2. The amino acid sequence set forth in SEQ ID NO: 10 is obtained by inserting two alanine residues at the C-terminal side of each (A)ₙ motif of the amino acid sequence set forth in SEQ ID NO: 4 and further substituting a part of glutamine (Q) residues with a serine (S) residue to delete a part of amino acids on the N-terminal side so as to be almost the same as the molecular weight of SEQ ID NO: 4. The amino acid sequence set forth in SEQ ID NO: 12 is an amino acid sequence in which a His tag has been added to the C-terminus of a sequence obtained by repeating, 4 times, the region of the 20 domain sequences present in the amino acid sequence set forth in SEQ ID NO: 9 (however, several amino acid residues at the C-terminal side of the region are substituted).

The value of x/y in the Giza ratio 1:1.8 to 1:11.3 of the amino acid sequence set forth in SEQ ID NO: 1 (corresponding to naturally occurring fibroin) is 15.0%. Values of x/y in the amino acid sequence set forth in SEQ ID NO: 2 and the amino acid sequence set forth in SEQ ID NO: 4 are both 93.4%. The value of x/y in the amino acid sequence set forth in SEQ ID NO: 10 is 92.7%. The value of x/y in the amino acid sequence set forth in SEQ ID NO: 12 is 89.3%. The values of z/w at the amino acid sequences set forth in SEQ ID NO: 1, SEQ ID NO: 2, SEQ ID NO: 4, SEQ ID NO: 10, and SEQ ID NO: 12 are respectively 46.8%, 56.2%, 70.1%, 66.1%, and 70.0%.

The modified fibroin of (3-i) may consist of an amino acid sequence set forth in SEQ ID NO: 2, SEQ ID NO: 4, SEQ ID NO: 10, or SEQ ID NO: 12.

The modified fibroin of (3-ii) includes an amino acid sequence having 90% or more sequence identity with the amino acid sequence set forth in SEQ ID NO: 2, SEQ ID NO: 4, SEQ ID NO: 10, or SEQ ID NO: 12. The modified fibroin of (3-ii) is also a protein including a domain sequence represented by Formula 1: [(A)ₙ motif-REP] m. The sequence identity is preferably 95% or more.

The modified fibroin of (3-ii) preferably has 90% or more sequence identity with the amino acid sequence set forth in SEQ ID NO: 2, SEQ ID NO: 4, SEQ ID NO: 10, or SEQ ID NO: 12, and has an amino acid sequence in which x/y is 64.2% or more, in the case where the number of amino acid residues in REPs of two adjacent [(A)ₙ motif-REP] units is sequentially compared from the N-terminal side to the C-terminal side, and the number of amino acid residues in REP having a smaller number of amino acid residues is defined as 1, the maximum value of the total value of the number of amino acid residues in the two adjacent [(A)ₙ motif-REP] units where the ratio of the number of amino acid residues in the other REP is 1.8 to 11.3 (a Giza ratio of 1:1.8 to 1:11.3) is defined as x, and the total number of amino acid residues of the domain sequence is y.

The above-mentioned modified fibroin may include the above-mentioned tag sequence at either or both of the N-terminus and C-terminus.

A more specific example of the modified fibroin including the tag sequence may be a modified fibroin including (3-iii) an amino acid sequence set forth in SEQ ID NO: 7, SEQ ID NO: 9, SEQ ID NO: 11, or SEQ ID NO: 13; or (2-iv) an amino acid sequence having 90% or more sequence identity with the amino acid sequence set forth in SEQ ID NO: 7, SEQ ID NO: 9, SEQ ID NO: 11, or SEQ ID NO: 13.

The amino acid sequences set forth in SEQ ID NO: 6, SEQ ID NO:7, SEQ ID NO: 8, SEQ ID NO: 9, SEQ ID NO: 11, and SEQ ID NO: 13 are amino acid sequences in which an amino acid sequence set forth in SEQ ID NO: 5 (including a His tag sequence) is added at the N-terminus of the amino acid sequences set forth in SEQ ID NO: 1, SEQ ID NO: 2, SEQ ID NO: 3, SEQ ID NO: 4, SEQ ID NO: 10, and SEQ ID NO: 12, respectively.

The modified fibroin of (3-iii) may consist of an amino acid sequence set forth in SEQ ID NO: 7, SEQ ID NO: 9, SEQ ID NO: 11, or SEQ ID NO: 13.

The modified fibroin of (3-iv) includes an amino acid sequence having 90% or more sequence identity with the amino acid sequence set forth in SEQ ID NO: 7, SEQ ID NO: 9, SEQ ID NO: 11, or SEQ ID NO: 13. The modified fibroin of (3-iv) is also a protein including a domain sequence represented by Formula 1: [(A)ₙ motif-REP]ₘ. The sequence identity is preferably 95% or more.

The modified fibroin of (3-iv) preferably has 90% or more sequence identity with the amino acid sequence set forth in SEQ ID NO: 7, SEQ ID NO: 9, SEQ ID NO: 11, or SEQ ID NO: 13, and has an amino acid sequence in which x/y is 64.2% or more, in the case where the number of amino acid residues in REPs of two adjacent [(A)ₙ motif-REP] units is sequentially compared from the N-terminal side to the C-terminal side, and the number of amino acid residues in REP having a smaller number of amino acid residues is defined as 1, the maximum value of the total value of the number of amino acid residues in the two adjacent [(A)ₙ motif-REP] units where the ratio of the number of amino acid residues in the other REP is 1.8 to 11.3 is defined as x, and the total number of amino acid residues of the domain sequence is y.

The above-mentioned modified fibroin may include a secretory signal for releasing the protein produced in the recombinant protein production system to the outside of a host. The sequence of the secretory signal can be appropriately set depending on the type of the host.

The modified fibroin in which a content of glycine residues and a content of (A)ₙ motifs are reduced is a modified fibroin in which the domain sequence has an amino acid sequence in which the content of glycine residues is reduced in addition to having a reduced content of (A)ₙ motifs as compared to naturally occurring fibroin. The domain sequence of the modified fibroin can be said to further have an amino acid sequence corresponding at least the substitution of one or a plurality of glycine residues in REP with another amino acid residue, in addition to deletion of one or a plurality of (A)ₙ motifs, as compared to naturally occurring fibroin. In other words, it is a modified fibroin having characteristics of the modified fibroin in which a content of glycine residues is reduced, and the modified fibroin in which a content of (A)ₙ motifs is reduced in combination. Specific aspects thereof are as described in the modified fibroin in which a content of glycine residues is reduced, and the modified fibroin in which a content of (A)ₙ motifs is reduced.

A more specific example of the modified fibroin in which a content of glycine residues and a content of (A)ₙ motifs are reduced may be a modified fibroin including (4-i) an amino acid sequence set forth in SEQ ID NO: 4, SEQ ID NO: 10, or SEQ ID NO: 12; or (4-ii) an amino acid sequence having 90% or more sequence identity with the amino acid sequence set forth in SEQ ID NO: 4, SEQ ID NO: 10, or SEQ ID NO: 12. Specific aspects of the modified fibroin including the amino acid sequence set forth in SEQ ID NO: 4, SEQ ID NO: 10, or SEQ ID NO: 12 are as described above.

The modified fibroin according to another embodiment may be a modified fibroin having an amino acid sequence having a region locally having a large hydrophobicity index, in which the domain sequence thereof corresponds to a sequence in which one or a plurality of amino acid residues in the REP is substituted by an amino acid residue having a large hydrophobicity index, and/or one or a plurality of amino acid residues having a large hydrophobicity index is inserted into the REP, as compared to naturally occurring fibroins.

The region locally having a large hydrophobicity index is preferably composed of 2 to 4 consecutive amino acid residues.

The amino acid residue having a large hydrophobicity index mentioned above is more preferably an amino acid residue selected from isoleucine (I), valine (V), leucine (L), phenylalanine (F), cysteine (C), methionine (M), and alanine (A).

In the modified fibroin according to the present embodiment may be a modified fibroin, in addition to modification corresponding to substitution of one or a plurality of amino acid residues in the REP by an amino acid residue having a large hydrophobicity index, and/or insertion of one or a plurality of amino acid residues having a large hydrophobicity index into the REP, as compared to naturally occurring fibroins, there may a modification of the amino acid sequence corresponding to substitution, deletion, insertion, and/or addition of one or a plurality of amino acid residues, as compared to naturally occurring fibroins.

The modified fibroin according to the present embodiment can be obtained by, from the gene sequence of cloned naturally occurring fibroins, substituting one or a plurality of hydrophilic amino acid residues in the REP (for example, amino acid residues whose hydrophobicity index is negative) by hydrophobic amino acid residues (for example, amino acid residues whose hydrophobicity index is positive); and/or by inserting one or a plurality of hydrophobic amino acid residues into REP. In addition, the modified fibroin according to the present embodiment can be obtained by, for example, designing an amino acid sequence corresponding to substitution of one or a plurality of hydrophilic amino acid residues in the REP by hydrophobic amino acid residues, from the amino acid sequence of naturally occurring fibroins, and/or insertion of one or a plurality of hydrophobic amino acid residues into the REP; and chemically synthesizing a nucleic acid encoding the designed amino acid sequence. In any case, in addition to modification corresponding to substitution of one or a plurality of hydrophilic amino acid residues in the REP by hydrophobic amino acid residues, from the amino acid sequence of naturally occurring fibroins, and/or insertion of one or a plurality of hydrophobic amino acid residues into the REP, further modification of the amino acid sequence corresponding to substitution, deletion, insertion and/or addition of one or a plurality of amino acid residues may be carried out.

The modified fibroin according to still another embodiment contains the domain sequence represented by Formula 1: [(A)ₙ motif-REP]ₘ and may have an amino acid sequence whose p/q is 6.2% or more, in all of the REP included in the sequence obtained by removing the sequence from the (A)ₙ motif located the most C-terminal side to the C-terminus of the domain sequence, from the above-mentioned domain sequence, in a case where the total number of amino acid residues contained in a region where the average value of the hydrophobicity index of four consecutive amino acid residues is 2.6 or more is p, and the total number of amino acid residues contained in the sequence obtained by removing the sequence from the (A)ₙ motif located the most C-terminal side to the C-terminus of the domain sequence, from the above-mentioned domain sequence is q.

With regard to the hydrophobicity index of amino acid residues, known indices (Hydropathy index: Kyte J, & Doolittle R (1982) “A simple method for displaying the hydropathic character of a protein,” J. Mol. Biol., 157, pp. 105-132) are used. Specifically, the hydrophobicity index (hydropathy index, hereinafter will be referred to as “HI”) of each amino acid is as shown in Table 1.

TABLE 1

| Amino acid | HI | Amino acid | HI |
|---|---|---|---|
| Isoleucine (He) | 4.5 | Tryptophan (Trp) | −0.9 |
| Valine (Val) | 4.2 | Tyrosine (Tyr) | −1.3 |
| Leucine (Leu) | 3.8 | Proline (Pro) | −1.6 |
| Phenylalanine (Phe) | 2.8 | Histidine (His) | −3.2 |
| Cysteine (Cys) | 2.5 | Asparagine (Asn) | −3.5 |
| Methionine (Met) | 1.9 | Asparaginic acid (Asp) | −3.5 |
| Alanine (Ala) | 1.8 | Glutamine (Gin) | −3.5 |
| Glycine (Gly) | −0.4 | Glutamic acid (Glu.) | −3.5 |
| Threonine (Thr) | −0.7 | Lysine (lys) | −3.9 |
| Serine (Ser) | −0.8 | Arginine (Arg) | −4.5 |

The calculation method of p/q will be described in more detail. In the calculation, the sequence obtained by removing the sequence from the (A)ₙ motif located at the most C-terminal side to the C-terminus of the domain sequence, from the domain sequence represented by Formula 1: [(A)ₙ motif-REP]ₘ, is used. First, in all REPs included in sequence A, an average value of the hydrophobicity index of four consecutive amino acid residues is calculated. The average value of the hydrophobicity index is obtained by dividing the sum of HI of each amino acid residue contained in the four consecutive amino acid residues by 4 (the number of amino acid residues). The average value of the hydrophobicity index is obtained for all four consecutive amino acid residues (each amino acid residue is used to calculate an average of 1 to 4 times). Next, a region in which the average value of the hydrophobicity index of the four consecutive amino acid residues is 2.6 or more is identified. Even in a case where a certain amino acid residue corresponds to the “four consecutive amino acid residues in which the average value of the plurality of hydrophobicity indices is 2.6 or more,” this amino acid residue is included in the region as one amino acid residue. In addition, a total number of amino acid residues contained in the region is p. Furthermore, the total number of amino acid residues contained in the sequence A is q.

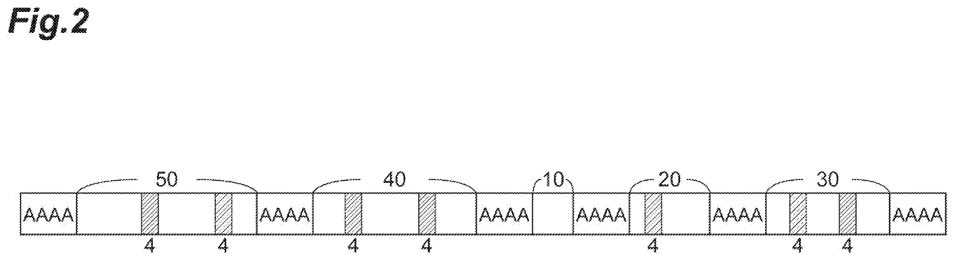

For example, in a case where consecutive four amino acid residues having the average value of the hydrophobicity index of 2.6 or more are extracted at 20 positions (no duplication), in the region where the average value of the hydrophobicity index of the four consecutive amino acid residues is 2.6 or more, there are 20 consecutive four amino acid residues (no duplication), and therefore p is 20×4=80. In addition, for example, in a case where two “consecutive four amino acid residues having an average value of the hydrophobicity index of 2.6 or more” overlap by one amino acid residue, in the region where the average value of the hydrophobicity index of the four consecutive amino acid residues is 2.6 or more, seven amino acid residues are contained (p=2×4−1=7, where “−1” is a subtraction of duplicates). For example, in the case of the domain sequence shown in FIG. 2, p is 7×4=28, because seven “consecutive four amino acid residues having the average value of the hydrophobicity index of 2.6 or more” are present without duplication. In addition, for example, in the case of the domain sequence shown in FIG. 2, q is 4+50+4+40+4+10+4+20+4+30=170 (not including the (A)ₙ motif present at the C-terminus). Next, p/q (%) can be calculated by dividing p by q. In the case of FIG. 2, 28/170=16.47%.

In the modified fibroin according to the present embodiment, p/q is preferably 6.2% or more, more preferably 7% or more, still more preferably 10% or more, even still more preferably 20% or more, and still further preferably 30% or more. The upper limit of p/q is not particularly limited, but it may be 45% or less, for example.

The modified fibroin according to the present embodiment can be obtained by modifying the amino acid sequence of cloned naturally occurring fibroins to the amino acid sequence containing the region locally having a large hydrophobicity index, by substituting one or a plurality of hydrophilic amino acid residues in the REP (for example, amino acid residues whose hydrophobicity index is negative) by hydrophobic amino acid residues (for example, amino acid residues whose hydrophobicity index is positive); and/or by inserting one or a plurality of hydrophobic amino acid residues into REP, such that the above conditions of p/q is satisfied. Alternatively, the modified fibroin according to the embodiment can also be obtained, for example, by designing an amino acid sequence satisfying the conditions of p/q from the amino acid sequence of naturally occurring fibroin and chemically synthesizing a nucleic acid encoding the designed amino acid sequence. In any case, in addition to modification corresponding to substitution of one or a plurality of amino acid residues in the REP by amino acid residues having a large hydrophobicity index, and/or insertion of one or a plurality of amino acid residues having a large hydrophobicity index into the REP as compared to naturally occurring fibroins, further modification corresponding to substitution, deletion, insertion and/or addition of one or a plurality of amino acid residues may be carried out.

The amino acid residue having a large hydrophobicity index mentioned above is not particularly limited, but is preferably isoleucine (I), valine (V), leucine (L), phenylalanine (F), cysteine (C), methionine (M), and alanine (A), and is more preferably valine (V), leucine (L), and isoleucine (I).

Other specific examples of the modified fibroin include modified fibroins including (5-i) an amino acid sequence set forth in SEQ ID NO: 19, SEQ ID NO: 21, or SEQ ID NO: 22, or (5-ii) an amino acid sequence having 90% or more sequence identity with the amino acid sequence set forth in SEQ ID NO: 19, SEQ ID NO: 21, or SEQ ID NO: 22.

The modified fibroin of (5-i) will be described. The amino acid sequence set forth in SEQ ID NO: 4 is an amino acid sequence in which consecutive amino acid sequences are deleted such that the number of consecutive alanine residues in the (A)ₙ motif of naturally occurring fibroins becomes 5. The amino acid sequence set forth in SEQ ID NO: 19 is an amino acid sequence in which, with respect to the amino acid sequence set forth in SEQ ID NO: 4, an amino acid sequence (VLI) consisting of three amino acid residues is inserted in two places every REP, respectively, and some of amino acids at the C-terminal side is deleted so that a molecular weight thereof becomes almost the same molecular weight as the amino acid sequence set forth in SEQ ID NO: 4. The amino acid sequence set forth in SEQ ID NO: 20 is obtained by inserting two alanine residues at the C-terminal side of each (A)ₙ motif with respect to the amino acid sequence set forth in SEQ ID NO: 19, and further substituting a part of glutamine (Q) residues with a serine (S) residue to delete a part of amino acids on the C-terminal side so as to be almost the same as the molecular weight of the amino acid sequence set forth in SEQ ID NO: 4. The amino acid sequence set forth in SEQ ID NO: 21 is an amino acid sequence in which, with respect to the amino acid sequence set forth in SEQ ID NO: 20, an amino acid sequence (VII) consisting of three amino acid residues is inserted in one place every REP, respectively. The amino acid sequence set forth in SEQ ID NO: 22 is an amino acid sequence in which, with respect to the amino acid sequence set forth in SEQ ID NO: 20, an amino acid sequence (VLI) consisting of three amino acid residues is inserted in two places every REP, respectively.

The modified fibroin of (5-i) may consist of the amino acid sequence set forth in SEQ ID NO: 19, SEQ ID NO: 21, or SEQ ID NO: 22.

The modified fibroin of (5-ii) includes an amino acid sequence having 90% or more sequence identity with the amino acid sequence set forth in SEQ ID NO: 19, SEQ ID NO: 21, or SEQ ID NO: 22. The modified fibroin of (5-ii) is also a protein including a domain sequence represented by Formula 1: [(A)ₙ motif-REP] m. The sequence identity is preferably 95% or more.

The modified fibroin of (5-ii) has 90% or more sequence identity with the amino acid sequence set forth in SEQ ID NO: 19, SEQ ID NO: 21, or SEQ ID NO: 22, and preferably has p/q of 6.2% or more, in all of the REP included in the sequence obtained by removing the sequence from the (A)ₙ motif located the most C-terminal side to the C-terminus of the domain sequence, from the above-mentioned domain sequence, in a case where the total number of amino acid residues contained in a region where the average value of the hydrophobicity index of four consecutive amino acid residues is 2.6 or more is p, and the total number of amino acid residues contained in the sequence obtained by removing the sequence from the (A)ₙ motif located the most C-terminal side to the C-terminus of the domain sequence, from the above-mentioned domain sequence is q.

The above-mentioned modified fibroin may include a tag sequence at either or both of the N-terminus and C-terminus.

A more specific example of the modified fibroin including a tag sequence may be a modified fibroin including (5-iii) an amino acid sequence set forth in SEQ ID NO: 23, SEQ ID NO: 24, or SEQ ID NO: 25, or (5-iv) an amino acid sequence having 90% or more sequence identity with the amino acid sequence set forth in SEQ ID NO: 23, SEQ ID NO: 24, or SEQ ID NO: 25.

The amino acid sequences set forth in SEQ ID NO: 23, SEQ ID NO:24, and SEQ ID NO: 25 are amino acid sequences in which an amino acid sequence set forth in SEQ ID NO: 5 (including a His tag sequence and a hinge sequence) is added at the N-terminus of the amino acid sequences set forth in SEQ ID NO: 19, SEQ ID NO: 21, and SEQ ID NO: 22, respectively.

The modified fibroin of (5-iii) may consist of an amino acid sequence set forth in SEQ ID NO: 23, SEQ ID NO: 24, or SEQ ID NO: 25.

The modified fibroin of (5-iv) includes an amino acid sequence having 90% or more sequence identity with the amino acid sequence set forth in SEQ ID NO: 23, SEQ ID NO: 24, or SEQ ID NO: 25. The modified fibroin of (5-iv) is also a protein including a domain sequence represented by Formula 1: [(A)ₙ motif-REP]ₘ. The sequence identity is preferably 95% or more.

The modified fibroin of (5-iv) has 90% or more sequence identity with the amino acid sequence set forth in SEQ ID NO: 23, SEQ ID NO: 24, or SEQ ID NO: 25, and preferably has p/q of 6.2% or more, in all of the REP included in the sequence obtained by removing the sequence from the (A)ₙ motif located the most C-terminal side to the C-terminus of the domain sequence, from the above-mentioned domain sequence, in a case where the total number of amino acid residues contained in a region where the average value of the hydrophobicity index of four consecutive amino acid residues is 2.6 or more is p, and the total number of amino acid residues contained in the sequence obtained by removing the sequence from the (A)ₙ motif located the most C-terminal side to the C-terminus of the domain sequence, from the above-mentioned domain sequence is q.

The above-mentioned modified fibroin may include a secretory signal for releasing the protein produced in the recombinant protein production system to the outside of a host. The sequence of the secretory signal can be appropriately set depending on the type of the host.

Examples of proteins derived from the weft protein include a protein including a domain sequence represented by Formula 3: [REP2]ₒ (where, in Formula 3, REP2 represents an amino acid sequence composed of Gly-Pro-Gly-Gly-X, X represents one amino acid selected from the group consisting of alanine (Ala), serine (Ser), tyrosine (Tyr), and valine (Val), and o represents an integer of 8 to 300). Specific examples thereof include a protein including the amino acid sequence set forth in SEQ ID NO: 26. The amino acid sequence set forth in SEQ ID NO: 26 is an amino acid sequence in which the amino acid sequence (referred to as a PR1 sequence) from 1220th residue to 1659th residue from the N-terminus corresponding to a motif and a repeat part of a partial sequence (NCBI Accession No.: AAF36090, GI: 7106224) of the flagelliform silk protein of Nephila clavipes obtained from the NCBI database is bonded to the C-terminal amino acid sequence from the 816th residue to 907th residue from the C-terminus of a partial sequence (NCBI Accession No.: AAC38847, GI: 2833649) of the flagelliform silk protein of Nephila clavipes obtained from the NCBI database; and the amino acid sequence (a tag sequence and a hinge sequence) set forth in SEQ ID NO: 5 is added to the N-terminus of the bonded sequence.

Examples of proteins derived from collagen include a protein including a domain sequence represented by Formula 4: [REP3] (where, in Formula 4, p represents an integer of 5 to 300, REP3 represents the amino acid sequence consisting of Gly-X-Y, X and Y represent any amino acid residues other than Gly, and a plurality of REP3's may be the same amino acid sequence with each other or different amino acid sequences from each other). Specific examples thereof include a protein including the amino acid sequence set forth in SEQ ID NO: 27. The amino acid sequence set forth in SEQ ID NO: 27 is an amino acid sequence in which the amino acid sequence (a tag sequence and a hinge sequence) set forth in SEQ ID NO: 5 is added to the N-terminus of the amino acid sequence from 301st residue to 540th residue corresponding to the motif and a repeat part of a partial sequence of human collagen type 4 obtained from the NCBI database (Accession No: CAA56335.1, GI: 3702452 of NCBI GenBank).

Examples of proteins derived from resilin include a protein including a domain sequence represented by Formula 5: [REP4]_{q} (where, in Formula 5, q represents an integer of 4 to 300; REP4 represents the amino acid sequence consisting of Ser-J-J-Tyr-Gly-U-Pro; J represents any amino acid residue, and is particularly preferably an amino acid residue selected from the group consisting of Asp, Ser, and Thr; U is any amino acid residue, and is particularly preferably an amino acid residue selected from the group consisting of Pro, Ala, Thr, and Ser; and a plurality of REP4's may be the same amino acid sequence with each other or different amino acid sequences from each other). Specific examples thereof include a protein including the amino acid sequence set forth in SEQ ID NO: 28. The amino acid sequence set forth in SEQ ID NO: 28 is an amino acid sequence in which the amino acid sequence (a tag sequence and a hinge sequence) set forth in SEQ ID NO: 5 is added at the N-terminus of the amino acid sequence from 19th residue to 321st residue of the sequence in which Thr at 87th position is replaced by Ser, and Asn at 95th position is replaced by Asp, in the amino acid sequence of resilin (Accession No. NP611157, GI: 24654243 of NCBI GenBank).

Examples of proteins derived from elastin include a protein having an amino acid sequence such as Accession Nos. AAC98395 (human), I47076 (sheep), NP786966 (bovine), and the like of GenBank of NCBI. Specific examples thereof include a protein including the amino acid sequence set forth in SEQ ID NO: 29. The amino acid sequence set forth in SEQ ID NO: 29 is an amino acid sequence in which the amino acid sequence set forth in SEQ ID NO: 5 (a tag sequence and a hinge sequence) is added to the N-terminus of the amino acid sequence from 121st residue to 390th residue of the amino acid sequence of Accession No. AAC98395 of GenBank of NCBI.

The structural protein described above and the protein derived from the structural protein can be used alone or in combination of two or more kinds thereof.

A protein fiber and a protein contained as a main component in a protein raw fiber can be produced by, for example, expressing the nucleic acid by a nucleic acid sequence encoding the protein, and a host transformed with an expression vector having one or a plurality of regulatory sequences operably linked to the nucleic acid sequence.

A method for producing a nucleic acid encoding the protein fiber and the protein contained in the protein raw fiber as the main component is not particularly limited. A nucleic acid can be produced by, for example, a method in which a gene encoding natural structural protein is amplified and cloned by polymerase chain reaction (PCR) or the like; or a method of chemically synthesizing a nucleic acid. A method for chemically synthesizing a nucleic acid is not particularly limited, and, for example, genes can be chemically synthesized by a method in which of linking, by PCR or the like, oligonucleotides that are automatically synthesized by AKTA oligopilot plus 10/100 (GE Healthcare Japan Ltd.) or the like, based on the amino acid sequence information of the structural protein obtained from the NCBI web database and the like. At this time, in order to facilitate purification and/or confirmation of the protein, a nucleic acid encoding the protein consisting of an amino acid sequence obtained by adding an amino acid sequence consisting of a start codon and a His10 tag to the N terminus of the above amino acid sequence may be synthesized.

The regulatory sequence is a sequence (for example, a promoter, an enhancer, a ribosome binding sequence, or a transcription termination sequence) that controls the expression of a recombinant protein in a host, and can be appropriately selected depending on the type of the host. As a promoter, an inducible promoter which functions in host cells and is capable of inducible expression of a target protein may be used. An inducible promoter is a promoter that can control transcription due to the presence of an inducer (expression inducer), the absence of a repressor molecule, or physical factors such as an increase or decrease in temperature, osmotic pressure, or pH value.

The type of the expression vector such as a plasmid vector, a viral vector, a cosmid vector, a fosmid vector, or an artificial chromosome vector can be appropriately selected depending on the type of the host. As the expression vector, an expression vector which can autonomously replicate in a host cell or can be incorporated into a chromosome of a host and which contains a promoter at a position capable of transcribing the nucleic acid encoding a target protein is suitably used.

Both prokaryotes and eukaryotes such as yeast, filamentous fungi, insect cells, animal cells, and plant cells can be suitably used as hosts.

Examples of hosts of the prokaryote include bacteria belonging to the genus Escherichia, Brevibacillus, Serratia, Bacillus, Microbacterium, Brevibacterium, Corynebacterium and Pseudomonas. Examples of microorganisms belonging to the genus Escherichia include Escherichia coli and the like. Examples of microorganisms belonging to the genus Brevibacillus include Brevibacillus agri and the like. Examples of microorganisms belonging to the genus Serratia include Serratia liquefaciens and the like. Examples of microorganisms belonging to the genus Bacillus include Bacillus subtilis and the like. Examples of microorganisms belonging to the genus Microbacterium include Microbacterium ammoniaphilum. Examples of microorganisms belonging to the genus Brevibacterium include Brevibacterium divaricatum and the like. Examples of microorganisms belonging to the genus Corynebacterium include Corynebacterium ammoniagenes and the like. Examples of microorganisms belonging to the genus Pseudomonas include Pseudomonas putida and the like.

In a case where a prokaryote is used as a host, examples of vectors into which a nucleic acid encoding a target protein is introduced include pBTrp2 (manufactured by Boehringer Mannheim), pGEX (manufactured by Pharmacia), pUC18, pBluescriptII, pSupex, pET22b, pCold, pUB110, and pNCO2 (Japanese Unexamined Patent Publication No. 2002-238569), and the like.

Examples of eukaryotic hosts include yeast and filamentous fungi (mold and the like). Examples of yeasts include a yeast which belongs to the genus Saccharomyces, Pichia, Schizosaccharomyces, and the like. Examples of filamentous fungi include filamentous fungi belonging to the genus Aspergillus, Penicillium, Trichoderma, and the like.

In a case where a eukaryote is used as a host, examples of vectors into which a nucleic acid encoding a target protein is introduced include YEP13 (ATCC37115), YEp24 (ATCC37051), and the like. As a method for introducing an expression vector into the foregoing host cell, any method can be used as long as it introduces DNA into the host cell. Examples thereof include a method using calcium ions [Proc. Natl. Acad. Sci. USA, 69, 2110 (1972)], an electroporation method, a spheroplast method, a protoplast method, a lithium acetate method, a competent method, and the like.

As a method for expressing a nucleic acid by a host transformed with an expression vector, secretory production, fusion protein expression, or the like, in addition to direct expression, can be carried out according to the method described in Molecular Cloning, 2nd edition.

The protein can be produced, for example, by culturing a host transformed with the expression vector in a culture medium, producing and accumulating the protein in the culture medium, and then collecting the protein from the culture medium. The method for culturing the host in a culture medium can be carried out according to a method commonly used for culturing a host.

In the case where the host is a prokaryote such as Escherichia coli or a eukaryote such as yeast, any of a natural medium and a synthetic medium may be used as a culture medium as long as it contains a carbon source, a nitrogen source, inorganic salts and the like which can be assimilated by the host and it is capable of efficiently culturing the host.

As the carbon source, any carbon source that can be assimilated by the transformed microorganism may be used. Examples of the carbon source that can be used include carbohydrates such as glucose, fructose, sucrose, and molasses, starch and starch hydrolyzates containing them, organic acids such as acetic acid and propionic acid, and alcohols such as ethanol and propanol. Examples of the nitrogen source that can be used include ammonium salts of inorganic or organic acids such as ammonia, ammonium chloride, ammonium sulfate, ammonium acetate and ammonium phosphate, other nitrogen-containing compounds, peptone, meat extract, yeast extract, corn steep liquor, casein hydrolyzate, soybean cake and soybean cake hydrolyzate, various fermented microbial cells and digested products thereof. As inorganic salts, it is possible to use potassium dihydrogen phosphate, dipotassium phosphate, magnesium phosphate, magnesium sulfate, sodium chloride, ferrous sulfate, manganese sulfate, copper sulfate, and calcium carbonate.

Culture of a prokaryote such as Escherichia coli or a eukaryote such as yeast can be carried out under aerobic conditions such as shaking culture or deep aeration stirring culture. The culture temperature is, for example, 15° C. to 40° C. The culture time is usually 16 hours to 7 days. It is preferable to maintain the pH of the culture medium during the culture at 3.0 to 9.0. The pH of the culture medium can be adjusted using an inorganic acid, an organic acid, an alkali solution, urea, calcium carbonate, ammonia, or the like.

In addition, antibiotics such as ampicillin and tetracycline may be added to the culture medium as necessary during the culture. In the case of culturing a microorganism transformed with an expression vector using an inducible promoter as a promoter, an inducer may be added to the medium as necessary. For example, in the case of culturing a microorganism transformed with an expression vector using a lac promoter, isopropyl-β-D-thiogalactopyranoside or the like is used, and in the case of culturing a microorganism transformed with an expression vector using a trp promoter, indole acrylic acid or the like may be added to the medium.

Isolation and purification of the expressed protein can be performed by a commonly used method. For example, in the case where the protein is expressed in a dissolved state in cells, the host cells are recovered by centrifugation after completion of the culture, suspended in an aqueous buffer solution, and then disrupted using an ultrasonicator, a French press, a Manton-Gaulin homogenizer, a Dyno-Mill, or the like to obtain a cell-free extract. From the supernatant obtained by centrifuging the cell-free extract, a purified preparation can be obtained by a method commonly used for protein isolation and purification, that is, a solvent extraction method, a salting-out method using ammonium sulfate or the like, a desalting method, a precipitation method using an organic solvent, an anion exchange chromatography method using a resin such as diethylaminoethyl (DEAE)-Sepharose or DIAION MPA-75 (manufactured by Mitsubishi Kasei Kogyo Kabushiki Kaisha), an cation exchange chromatography method using a resin such as S-Sepharose FF (Pharmacia Corporation), a hydrophobic chromatography method using a resin such as butyl sepharose or phenyl sepharose, a gel filtration method using a molecular sieve, an affinity chromatography method, a chromatofocusing method, an electrophoresis method such as isoelectric focusing or the like, alone or in combination thereof.

In the case where the protein is expressed by the formation of an insoluble matter in the cell, similarly, the host cells are recovered, disrupted and centrifuged to recover the insoluble matter of the protein as a precipitated fraction. The recovered insoluble matter of the protein can be solubilized with a protein denaturing agent. After this operation, a purified preparation of the protein can be obtained by the same isolation and purification method as described above. In the case where the protein is secreted extracellularly, the protein can be recovered from the culture supernatant. That is, a culture supernatant is obtained by treating the culture by a technique such as centrifugation, and a purified preparation can be obtained from the culture supernatant by using the same isolation and purification method as described above.

(Protein Raw Fiber)

Protein raw fiber is obtained by spinning the above-described protein, and contains the above-described protein as a main component. The protein raw fiber can be produced by a known spinning method. That is, for example, in a case of producing the protein raw fiber containing the spider silk fibroin as a main component, first, a dope solution is prepared by adding and dissolving the spider silk fibroin produced according to the method described above in a solvent such as dimethyl sulfoxide (DMSO), N,N-dimethylformamide (DMF), hexafluoroisopronol (HFIP), or formic acid, together with inorganic salt as a dissolution promoter. Next, using this dope solution, spinning is performed by a known spinning method such as wet-type spinning, dry-type spinning, or dry-wet-type spinning, and thereby a target protein raw fiber can be obtained.

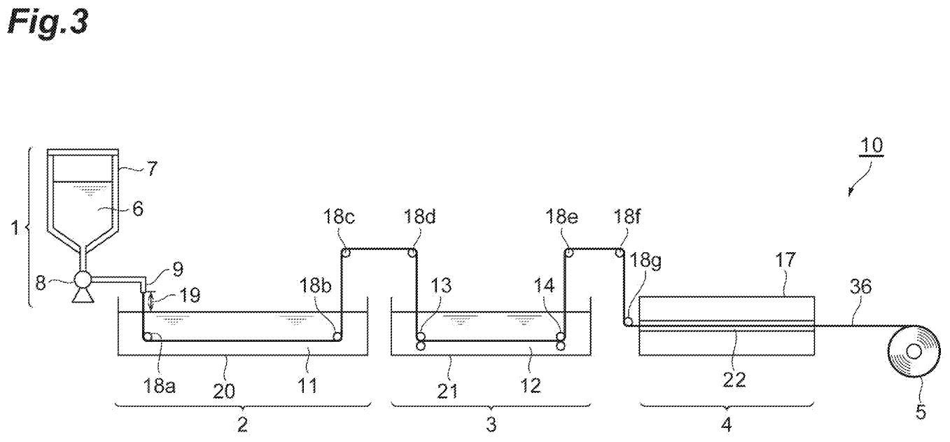

FIG. 3 is a schematic view showing an example of a spinning apparatus for producing protein raw fibers. A spinning apparatus 10 shown in FIG. 3 is an example of a spinning apparatus for dry-wet-type spinning, and has an extrusion apparatus 1, a coagulation bath 20, a washing bath 21, and a drying apparatus 4 in this order from the upstream side.

The extrusion apparatus 1 has a storage tank 7, in which a dope solution (spinning undiluted solution) 6 is stored. A coagulation liquid 11 (for example, methanol) is stored in the coagulation bath 20. The dope solution 6 is pushed out from a nozzle 9 provided by opening an air gap 19 between the dope solution 6 and the coagulation liquid 11, by a gear pump 8 attached to a lower end of the storage tank 7. The extruded dope solution 6 is supplied into the coagulation liquid 11 through the air gap 19. The solvent is removed from the dope solution 6 in the coagulation liquid 11 to coagulate the protein. The coagulated protein is guided to the washing bath 21 and washed with a washing solution 12 in the washing bath 21, and then sent to the drying apparatus 4 by a first nip roller 13 and a second nip roller 14 installed in the washing bath 21. At this time, for example, in a case where a rotational speed of the second nip roller 14 is set to be faster than a rotational speed of the first nip roller 13, protein raw fibers 36 drawn at a magnification corresponding to the rotational speed ratio is obtained. The protein raw fibers drawn in the washing solution 12 are separated from the inside of the washing bath 21 and then is dried when passing through the drying apparatus 4. Thereafter, the fibers are wound up by a winder. Accordingly, the protein raw fibers are obtained as a wound product 5 which is finally wound around the winder, by the spinning apparatus 10. 18a to 18g are yarn guides.

The coagulation liquid 11 may be a solution capable of desolvation, and examples thereof include lower alcohols having 1 to 5 carbon atoms such as methanol, ethanol and 2-propanol, and acetone. The coagulation liquid 11 may appropriately contain water. The temperature of the coagulation liquid 11 is preferably 0° C. to 30° C. The distance the coagulated protein passes in the coagulation liquid 11 (substantially, the distance from the yarn guide 18a to the yarn guide 18b) may be any length that enables efficient desolvation, and is, for example, 200 to 500 mm. The residence time in the coagulation liquid 11 may be, for example, 0.01 to 3 minutes and preferably 0.05 to 0.15 minutes. Further, drawing (pre-drawing) may be carried out in the coagulation liquid 11.

The drawing performed in the washing bath 21 when obtaining the protein raw fibers may be so-called wet heat drawing performed in warm water, in a solution in which an organic solvent or the like is added to warm water, or the like. A temperature of the wet heat drawing may be, for example, 50° C. to 90° C., and is preferably 75° C. to 85° C. In wet heat drawing, undrawn yarn (or pre-drawn yarn) can be drawn, for example, 1 time to 10 times, preferably 2 to 8 times.

The lower limit of the final draw ratio is preferably more than 1 time, 2 times or more, 3 times or more, 4 times or more, 5 times or more, 6 times or more, 7 times or more, 8 times or more, or 9 times or more a draw ratio of the undrawn yarn (or pre-drawn yarn). The upper limit is preferably 40 times or less, 30 times or less, 20 times or less, 15 times or less, 14 times or less, 13 times or less, 12 times or less, 11 times or less, or 10 times or less.

(Water Vapor Heat Treatment Step)

A water vapor heat treatment step is a step of bringing a protein raw fiber containing a protein into contact with water vapor in a storing chamber in which a temperature is adjusted within a range of less than 120° C. to heat-treat the protein raw fiber (a so-called “steam setting”). The protein raw fibers are contracted by a predetermined amount during the water vapor heat treatment (primary contraction), and is contracted also at the time of drying after the water vapor heat treatment (secondary contraction). In the protein raw fibers which have undergone such a water vapor heat treatment step, and furthermore, in the protein raw fibers which have been dried after the water vapor heat treatment, a contraction amount in a case of contact with water such as water, hot water, and water vapor is sufficiently reduced.

Specifically, for example, in the water vapor heat treatment step, while incorporating the protein raw fibers in a predetermined storing chamber, a temperature in the storing chamber is adjusted to within a range less than 120° C. by introducing water vapor into the storing chamber, and the protein raw fibers are brought into contact with water vapor to perform heat treatment of the protein raw fibers. The protein raw fibers to be subjected to the water vapor heat treatment step may be a bundle of a plurality of (for example, 5, 10, 20) spun proteins.

In a case of the water vapor heat treatment, when the temperature in the storing chamber reaches 120° C. or higher, the risk of the treatment operation increases and the workability also decreases. The temperature in the storing chamber is preferably 110° C. or less, and is more preferably 100° C. or less, from the viewpoint of avoiding the risk. The lower limit of the temperature in the storing chamber is not particularly limited as long as water vapor is brought into contact with the protein raw fibers in the storing chamber, but from the viewpoint that the effects of the present invention can be more remarkably obtained, the lower limit of the temperature is preferably any one of 50° C. or more, 60° C. or more, 70° C. or more, 80° C. or more, or 90° C. or more. Meanwhile, a temperature of water vapor to be brought into contact with the protein raw fibers during the water vapor heat treatment is not particularly limited, but from the viewpoint that the effects of the present invention can be more remarkably obtained, the lower limit thereof is any one of 60° C. or more, 70° C. or more, 80° C. or more, 90° C. or more, or 100° C. or more. From the same viewpoint, and from the viewpoint of safely performing the water vapor heat treatment, the upper limit of the temperature of the water vapor is preferably 120° C. or less, and is more preferably 110° C. or less.

A time for subjecting the protein raw fibers to the water vapor heat treatment is not particularly limited, and may be, for example, one minute or longer. The time may be 10 minutes or longer, may be 20 minutes or longer, and may be 30 minutes or longer. Further, the upper limit of the time is not particularly limited, but from the viewpoint of shortening the time of the production step and from the viewpoint of eliminating a possibility of hydrolysis of the protein raw fibers, the upper limit may be, for example, 120 minutes or shorter, may be 90 minutes or shorter, or may be 60 minutes or shorter.

The water vapor heat treatment (steam setting) can be performed by, for example, using a general steam setting device. Specific examples of steam setting devices include product name: FMSA-type steam setter (manufactured by Fukushin Kogyo Co., Ltd.), product name: EPS-400 (manufactured by Kasai Denki Kogyo Co., Ltd.), and the like.

The water vapor heat treatment may be performed under normal pressure or under reduced pressure (for example, vacuum steam setting).

In addition, the protein raw fibers to be subjected to the water vapor heat treatment may be pre-twisted. Accordingly, it is not necessary to carry out a twisting step with a steam set separately from the water vapor heat treatment for pre-shrinkage. Therefore, the production step of a target protein fiber is simplified, and it is also possible to advantageously suppress the damage to the protein fiber by redundant execution of the steam set.

In the water vapor heat treatment, in a case where the protein raw fibers are brought into contact with water vapor in a loosened state, the protein raw fibers may be crimped in a wavelike manner. In order to prevent the occurrence of such crimp, for example, the heat treatment may be carried out in a state where the protein raw fibers are not loosened, by bringing the protein raw fibers into contact with water vapor while stretching (stretching) them in a fiber axial direction. Examples of methods of not loosening the protein raw fibers include a method of applying load by suspending weight on protein raw fibers, and the like; a method for fixing both ends of protein raw fibers; and a method of winding protein raw fibers on a wound body such as paper tube or bobbin.

[Method for Pre-Shrinking Protein Fiber]

The method for producing a protein fiber of the present invention described above can be perceived as the method for pre-shrinking a protein fiber, including a step of storing protein raw fibers containing a protein in a storage chamber, and then adjusting a temperature of the storage chamber to less than 120° C. by introducing water vapor into the storage chamber, and thereby heat-treating the protein raw fibers.

[<Method for Producing Fabric Made of Protein Fibers]

The present invention also includes a method for producing fabric made of protein fibers including a step of producing fabric using the protein fiber obtained by the method for producing a protein fiber according to the present invention. The method for producing fabric from the protein fibers is not particularly limited, and known methods can be used.

According to the method for producing fabric made of protein fibers according to the present embodiment, it is possible to easily produce fabric made of the protein fibers in which a contraction amount due to contact with water is reduced, by using the protein fibers which have been subjected to the water vapor heat treatment step (the steam setting step) as described above.

Protein fibers used for producing the fabric made of the protein fibers may be short fibers or long fibers. In addition, such protein fibers may be used alone or in combination with other fibers. In other words, in a case of producing the fabric made of the protein fibers, as material yarns, a single yarn consisting only of protein fibers that have been subjected to the water vapor heat treatment step (the steam setting step), and a composite yarn formed by combining protein fibers subjected to the water vapor heat treatment step (the steam setting step) with other fibers may be used alone, or may be used in combination thereof. In addition, the other fibers mean protein fibers which have not been subjected to the water vapor heat treatment step (the steam setting step), fibers not containing a protein, and the like. Furthermore, examples of composite yarns include blended yarns, mixed yarns, covering yarns, and the like.

The type of fabric made of the protein fibers produced according to the method for producing fabric made of protein fibers according to the present embodiment is also not particularly limited. The fabric made of the protein fibers may be, for example, a woven or knitted fabric, or may be a non-woven fabric. In addition, the woven fabric may be, for example, fabric in which a woven structure is plain weave, twill weave, satin weave, and the like, and the type of yarn used may be one kind or plural kinds. The knitted fabric may be, for example, a warp knitted fabric such as tricot or russell, may be a weft knitted fabric such as a weft knitted fabric or a circular knitted fabric, and the type of yarn used may be one kind or plural kinds.

### EXAMPLES

Hereinafter, the present invention will be described more specifically with respect to Examples. However, the present invention is not limited to the following Examples.

[Production of Protein Raw Fibers]

<(1) Production of Spider Silk Protein (Spider Silk Fibroin: PRT799)>

(Synthesis of Gene Encoding Spider Silk Protein and Construction of Expression Vector)

Based on the base sequence and amino acid sequence of a fibroin (GenBank Accession Number: P46804.1, GI: 1174415) derived from Nephila clavipes, a modified fibroin (hereinafter, will be referred to as “PRT799”) having an amino acid sequence set forth in SEQ ID NO: 13 was designed.

With respect to the amino acid sequence of the fibroin derived from Nephila clavipes, the amino acid sequence set forth in SEQ ID NO: 13 has an amino acid sequence in which amino acid residues are substituted, inserted, and deleted for the purpose of improving productivity, and has the N-terminus to which the amino acid sequence (a tag sequence and a hinge sequence) set forth in SEQ ID NO: 5 is added.

Next, a nucleic acid encoding PRT799 was synthesized. In the nucleic acid, an NdeI site was added to the 5′ end and an EcoRI site was added downstream of the stop codon. The nucleic acid was cloned into a cloning vector (pUC118). Thereafter, the same nucleic acid was cleaved by restriction enzyme treatment with NdeI and EcoRI, and then recombined into a protein expression vector pET-22b(+) to obtain an expression vector.

Escherichia coli BLR (DE3) was transformed with the obtained expression vector pET22b (+) containing the nucleic acid encoding PRT799. The transformed Escherichia coli was cultured in 2 mL of an LB medium containing ampicillin for 15 hours. The culture solution was added to 100 mL of a seed culture medium (Table 2) containing ampicillin so that the OD₆₀₀ was 0.005. The temperature of the culture solution was maintained at 30° C. and the flask culture was carried out (for about 15 hours) until the OD₆₀₀ reached 5, thereby obtaining a seed culture solution.

TABLE 2
Seed culture medium

| Reagents | Concentration (g/L) |
|---|---|
| Glucose | 5.0 |
| KH₂PO₄ | 4.0 |
| K₂HPO₄ | 9.3 |
| Yeast Extract | 6.0 |
| Ampicillin | 0.1 |

The seed culture solution was added to a jar fermenter to which 500 ml of a production medium (Table 3) had been added so that the OD₆₀₀ was 0.05. The culture was carried out while maintaining the culture solution temperature at 37° C. and keeping the pH constant at 6.9. Further, the dissolved oxygen concentration in the culture solution was maintained at 20% of the dissolved oxygen saturation concentration.

TABLE 3
Production medium

| | Concentration | |
|---|---|---|
| Reagents | (g/L) | |
| Glucose | 12.0 | |
| KH₂PO₄ | 9.0 | |
| MgSO₄ · 7H₂O | 2.4 | |
| Yeast Extract | 15 | |
| FeSO₄ · 7H₂O | 0.04 | |
| MnSO₄ · 5H₂O | 0.04 | |
| CaCl₂ · 2H₂O | 0.04 | |
| ADEKANOL (LG-295S, Adeka Corporation) | 0.1 | (mL/L) |

Immediately after glucose in the production medium was completely consumed, a feed solution (455 g/1 L of glucose and 120 gill of Yeast Extract) was added at a rate of 1 mL/min. The culture was carried out while maintaining the culture solution temperature at 37° C. and keeping the pH constant at 6.9. Further, the dissolved oxygen concentration in the culture solution was maintained at 20% of the dissolved oxygen saturation concentration, and the culture was carried out for 20 hours. Thereafter, 1 M isopropyl-β-thiogalactopyranoside (IPTG) was added to the culture solution to a final concentration of 1 mM to induce the expression of PRT799. Twenty hours after addition of IPTG, the culture solution was centrifuged to recover the bacterial cells. SDS-PAGE was carried out using the bacterial cells prepared from the culture solution before the addition of IPTG and after the addition of IPTG, and the expression of PRT799 was confirmed by the appearance of a band of a size of PRT799 depending on the addition of IPTG.

(Purification of PRT799)

The bacterial cells recovered 2 hours after the addition of IPTG were washed with 20 mM Tris-HCl buffer solution (pH 7.4). The bacterial cells after washing were suspended in 20 mM Tris-HCl buffer solution (pH 7.4) containing about 1 mM PMSF, and the cells were disrupted with a high-pressure homogenizer (available from GEA Niro Soavi SpA). The disrupted cells were centrifuged to obtain a precipitate. The obtained precipitate was washed with 20 mM Tris-HCl buffer solution (pH 7.4) until high purity. The precipitate after washing was suspended in 8 M guanidine buffer solution (8 M guanidine hydrochloride, 10 mM sodium dihydrogen phosphate, 20 mM NaCl, 1 mM Tris-HCl, pH 7.0) so as to have a concentration of 100 mg/mL, and dissolved by stirring with a stirrer at 60° C. for 30 minutes. After dissolution, dialysis was carried out with water using a dialysis tube (cellulose tube 36/32 manufactured by Sanko Junyaku Co., Ltd.). The white aggregated protein (PRT799) obtained after dialysis was recovered by centrifugation, the water content was removed with a freeze dryer, and the freeze-dried powder was recovered.

<(2) Production of Protein Raw Fibers>

(Preparation of Dope Solution)

After adding spider fibroin (PRT799) mentioned above such that a concentration became 24% by mass to dimethyl sulfoxide (DMSO), a 4.0% by mass concentration of LiCl was added thereto as a dissolution promoter. Thereafter, the mixture was dissolved for 3 hours using a shaker. Thereafter, dust and bubbles were removed to obtain a dope solution. A solution viscosity of the dope solution was 5000 cP (centipoise) at 90° C.

(Spinning)

Using the dope solution obtained as described above and the spinning apparatus 10 shown in FIG. 3, known dry-wet-type spinning was performed to obtain protein raw fibers. The dry-wet-type spinning was performed under the following conditions.

Extrusion nozzle diameter: 0.1 mm

Coagulation liquid (methanol) temperature: 2° C.

Draw ratio: 4.52 times

Drying temperature: 80° C.

### Test Example 1: Production of Protein Fiber (1)—Normal Pressure Steam Set

The protein raw fibers obtained as described above were cut into 25 cm, and ten fibers were bundled to prepare two fiber bundles. Next, a 120 g weight was hung on one of the two fiber bundles, and in this state, the fiber bundle was set in a steam setting device (product name: Aoi Dyeing Machine Industry, Model No. EPS-400, manufactured by Aoi Dyeing Machine Industry Co., Ltd.). The water vapor heat treatment (steam setting) was performed under the conditions of normal pressure and a temperature of 85° C. in the steam setting device for 30 minutes. Next, the fiber bundle after the water vapor heat treatment was air dried. Thereby, protein fibers on which water vapor heat treatment was performed in a non-loosened state was obtained (Example 1). In addition, the water vapor heat treatment was performed on another fiber bundle in the same manner as described above, with the weight not suspended. Next, the fiber bundle after the water vapor heat treatment was air dried. Thereby, protein fibers on which the water vapor heat treatment was performed in a state where loosening was allowed was obtained (Example 2). When lengths of the protein fibers of Example 1 and Example 2 were measured, and the length of the protein fibers of Example 1 was 19.6 cm, and the length of the protein fibers of Example 2 was 17.3 cm. The fact that the protein fibers of Example 1 are longer than the protein fibers of Example 2 is considered to be because contraction of the protein fibers of Example 1 during the water vapor heat treatment is suppressed by the weight load.

Next, treatment (water contraction treatment) in which each of the protein fibers of Example 1 and Example 2 obtained as described above were immersed in water at 19° C. for 180 seconds and then naturally dried at a temperature of 20° C. and a relative humidity of 65% RH was performed. Thereafter, the lengths of the protein fibers of Examples 1 and 2 subjected to the water contraction treatment were each measured.

In addition, for comparison, protein (raw material) fibers obtained as above, which had only been cut to 25 cm, and which had not been subjected to the water vapor heat treatment, were used as Comparative Example 1. The same water contraction treatment as described above was performed on the protein fibers of Comparative Example 1. Thereafter, a length of the protein fibers of Comparative Example 1 subjected to the water contraction treatment was measured.

In addition, a contraction percentage of each of the protein fibers of Examples 1 and 2 and Comparative Example 1 in which the water contraction treatment was performed was calculated according to Equation 6.

Contraction percentage=(length before water contraction treatment−length after water contraction treatment)/length before water contraction treatment×100 Formula 6

As a result, the contraction percentage of the protein fibers of Example 1 produced by undergoing the water vapor heat treatment step (steam setting step) according to the present invention was 18.8%, and the contraction percentage of the protein fibers of Example 2 was 0%. In addition, no crimp was observed in the protein fibers of Example 1. On the other hand, the contraction percentage of the protein fibers of Comparative Example 1 which was not subjected to the water vapor heat treatment was 44%. Based on these results, it is clearly recognized that, although the contraction amounts differ depending on whether or not a load is applied during the water vapor heat treatment, the production method according to the present invention can produce protein fibers in which a contraction amount due to contact with moisture is reduced.

### Test Example 2: Production of Protein Fibers (2)—Pressure-Reduced Steam Set

In addition to Examples 1 and 2, the protein raw fibers obtained as described above were cut into 25 cm, and a fiber bundle of ten fibers was produced. Next, the water vapor heat treatment was performed on the fiber bundle as follows. In other words, using a steam setting device (product name: FMSA-type steam setter, manufactured by Fukushin Kogyo Co., Ltd.), the water vapor heat treatment (pressure-reduced steam set) was performed under the conditions of a temperature of 95° C. in the steam setting device for 30 minutes while reducing pressure. Next, the fiber bundle after water vapor heat treatment was air-dried and then allowed to stand overnight under conditions of a temperature of 20° C. and a relative humidity of 40% RH. Accordingly, protein fibers subjected to the water vapor heat treatment under reduced pressure were obtained (Example 3). A length of the protein fibers of Example 3 was 23.7 cm.

In the case of performing the water vapor heat treatment, the following operation was performed so that a temperature in the steam setting device was maintained at about 95° C. In other words, while a gas in the steam setting device was continuously suctioned by a suction device, water vapor was introduced into the device when a vacuum gauge exceeded 9333 Pa (70 mm Hg), and the temperature in the device was raised. When the temperature in the device reached 95° C., the introduction of water vapor into the device was once stopped. Then, when the temperature in the device was lowered by 3° C. from that state, water vapor was introduced into the device again. When the temperature in the device reached 95° C., the introduction of water vapor into the device was stopped. By repeating this operation, the temperature in the device was maintained at about 95° C.

Next, water contraction treatment using water vapor was performed on the protein fibers of Example 3 obtained as described above. In other words, as the water contraction treatment, a method was adopted in which the protein fibers of Example 3 were subjected to steam setting under the conditions of 90° C. for 30 minutes under normal pressure with the above-described steam setting device, and then air dried. Thereafter, after measuring a length of the protein fibers of Example 3 which underwent the water contraction treatment, a contraction percentage thereof was calculated according to Equation 6.

As a result, the contraction percentage of the protein fibers of Example 3 produced by undergoing the water vapor heat treatment step according to the present invention was 5.3%. Based on these results, it is clearly recognized that the production method according to the present invention which includes the step of performing the water vapor heat treatment under reduced pressure can produce protein fibers in which a contraction amount due to contact with moisture is reduced.

### REFERENCE SIGNS LIST

1: extrusion apparatus, 4: drying apparatus, 6: dope solution, 10: spinning apparatus, 20: coagulation bath, 21: washing bath

1. A method for producing a protein fiber, comprising a step of bringing a protein raw fiber containing a protein into contact with water vapor in a storing chamber in which a temperature is adjusted within a range of less than 120° C. to perform heat treatment of the protein raw fiber.

2. The method for producing a protein fiber according to claim 1, wherein the heat treatment is performed for 1 minute or longer.

3. The method for producing a protein fiber according to claim 1, wherein the protein is a structural protein.

4. The method for producing a protein fiber according to claim 3, wherein the structural protein is a spider silk fibroin.

5. The method for producing a protein fiber according to claim 1, wherein a plurality of the protein raw fibers are bundled together and are twisted.

6. The method for producing a protein fiber according to claim 1, wherein the heat treatment is performed in a state in which the protein raw fiber is not loosened.

7. The method for producing a protein fiber according to claim 1, wherein the heat treatment is performed under reduced pressure.

8. A method for producing fabric made of protein fibers, comprising a step of producing fabric using the protein fiber obtained by the method for producing a protein fiber according to claim 1.

9. A method for pre-shrinking a protein fiber, comprising a step of bringing a protein raw fiber containing a protein into contact with water vapor in a storing chamber in which a temperature is adjusted within a range of less than 120° C. to heat-treat the protein raw fiber.
